(19) United States
(12) Patent Application Publication
(10) Pub. No.: US 2024/0008812 A1
(43) Pub. Date: Jan. 11, 2024

(54) WEARABLE DATA COLLECTION DEVICE WITH NON-INVASIVE SENSING

(71) Applicant: SOBR SAFE, INC., Greenwood Village, CO (US)

(72) Inventors: Katia Marguerite BENSON, Greenwood Village, CO (US); Kevin Duane MOORE, Greenwood Village, CO (US); Scott BENNETT, Greenwood Village, CO (US)

(21) Appl. No.: 18/251,567

(22) PCT Filed: Nov. 3, 2021
(86) PCT No.: PCT/US2021/072203

§ 371 Date: May 3, 2023

Related U.S. Application Data
(60) Provisional application No. 63/109,134, filed on Nov. 3, 2020.

(51) Int. Cl. A61B 5/00; A61B 5/117; A61B 5/145

(52) U.S. Cl. CPC A61B 5/4845; A61B 5/681; A61B 5/117; A61B 5/14517; A61B 5/14546; A61B 5/6831

(57) ABSTRACT

Described is a wrist wearable device with non-invasive sensing for the detection, prediction, screening, abstention, or treatment of alcohol, drug use or abuse. In one embodiment, a device (100) includes a substance sensor (108) supported by the device (100). The substance sensor (108) may be any suitable sensor. As show, the device (100) is a wrist wearable device with a case (102), an attachment (104), and a substance sensor (108) supported by the case (102). The attachment (104) as shown is a one-piece band with a coupler (112). The device (100) may communicate with a mobile device such as a phone and, in turn, with a remote processing platform. The remote processing platform can use information from the device (100) for identification of the subject and detection of a condition of the subject in relation to consumption of alcohol or us of other substances, among other things.

### CROSS REFERENCE

This application claims priority to U.S. Provisional Application No. 63/109,134 filed Nov. 3, 2020, the entire contents of which are incorporated herein by reference.

### BACKGROUND OF THE INVENTION

### Field of the Invention

The present invention generally relates to wearable devices and more particularly to wearable devices for the identification of individuals and/or the screening, prediction or monitoring of substance use and physiology of human subjects.

### Description of the Background

Alcohol detection in human subjects is generally known, see for example US patent applications: 20130035602; and U.S. Pat. Nos. 3,764,270; 3,831,707; 3,815,087; 3,904,251; 4,613,845; 4,738,333; 4,749,553; 4,843,377; 4,914,038; 5,220,919; 5,944,661; 6,075,444; 6,229,908; 6,620,108; 7,311,665; 7,377,186; 7,616,123; 8,795,484; 9,296,298; 9,784,708 and Japanese publications: JP4940350B2; JP2004169524A2, the disclosures of which are incorporated herein by reference in their entirety.

Despite the vast amount of work done in the field, it has been found that wrist wearables have difficulty accurately detecting alcohol and other substances of abuse in individuals across a wide population under varied environmental and/or subject matter conditions. Therefore, there is a need for a wrist wearable device that can better assist in the detection, prediction, screening, abstention, and/or treatment of alcohol and drug abuse.

### SUMMARY OF THE INVENTION

Disclosed herein is a wearable device with one or more sensors and associated processing platforms and functionality. The wearable device may be a wrist worn wearable device or wrist wearable. Sensors may be positioned about the wearable device to measure one or more characteristics about a subject individual including an individual's substance use, predicted use, physiology, pathology, physical condition, mental condition, environmental surroundings, jitter, fine motor movements, and gross motor movements. The characteristics may be processed to provide reports, for example, to the subject, the subject's employer, a caregiver or support person of the subject, or an insurance company or regulator, among others. The benefits and solutions can be varied and numerous. One such solution can prioritize prevention over reaction.

In one set of embodiments there is provided a wearable device that can assist in managed care, telehealth, and/or treatment of substance abuse or addiction for individuals. Data collected from a wearable may be used to assist in the treatment of substance addiction, reduce the length of time for recovery, and/or reduce the time to intervention or receiving treatment. The wearable may be used to predict a person's potential for impending relapse.

In another set of embodiments there is provided a wearable device that can be used as a deterrent to working impaired. These embodiments may help to reduce workplace accidents related to substance use, fatigue, and stress. Certain embodiments may help change workplace behaviors and/or societal mind sets. The wearable may be worn by drivers, machine operators, and those with positions where a clear mind is needed for the safety of persons, property and the environment.

In another set of embodiments there is provided a wearable device tied to a data collection system. Data from one or more wearable devices may be used for predictive analytics. The wearable device may communicate data to a remote reporting system. A remote reporting system may assist health care providers in helping individuals through managed care, telehealth and substance abuse intervention. A remote reporting system can empower the use of data to provide decision makers with increased transparency into their organizations, customers, and clients.

In accordance with one aspect of the present invention, a system and associated functionality (“utility”) is provided for use in monitoring subjects. The utility involves user equipment including a wearable sensor device associated with a network interface device for enabling messaging between the wearable sensor device and a remote processing platform. The network interface device may be incorporated into the wearable and/or may include a mobile data device such as a phone or tablet computer. The remote processing platform may include a data processing system of the user, for example, in the case of a hosted application, or may include a cloud-based processing platform. The remote processing platform is operative to receive, from the subject equipment, at least first identification information concerning a first subject of the subject equipment and to receive sensor information for the first subject.

The remote processing platform can then process the sensor information to make a first determination concerning a condition of the first subject in relation to one of alcohol consumption and use of another substance such as a controlled substance. In this regard, the remote processing platform may perform a first identification of the subject based on static biometric information such as a fingerprint or facial identification information. Additionally or alternatively, the remote processing platform may make an identification of the subject based on dynamic biometric information such as heart activity or a pulsatile waveform of the subject. The remote processing platform may further be operative for verifying that the sensor device is being worn by the user and performing a liveness determination. The sensor information may be processed by a machine learning tool, for example, employing artificial intelligence. Based on the determination, the processing platform provides a report to a user concerning the condition of the first subject. The user may be, for example, the subject, the subject's employer, the subject's parent, a caregiver or support person of the subject, or an insurance company or regulator, among others. As described below, such users may receive information concerning alcohol consumption or use of other substances, location information (e.g., a graphical identification of the subject's current location), and other information.

The invention encompasses various embodiments of the subject equipment, various implementations of the remote processing platform, combinations of the subject equipment and the processing platform, and associated functionality.

For a more complete understanding of the claimed invention(s), reference is now made to the accompanying drawings and detailed description of preferred embodiments. Throughout the several figures and views, like symbols refer to like elements. It should also be noted that for method steps, unless specifically designated or limited by impossibility, steps may be performed in any order.

### BRIEF DESCRIPTION OF THE DRAWINGS

FIGS. 1A-1E show perspective views of an embodiment of a wearable device having a sensor.

FIG. 2 is an exploded view of an embodiment of a wearable device having a sensor.

FIG. 3 is an exploded view of an embodiment of a wearable device having a sensor.

FIG. 4 is a perspective view of an embodiment of a sensor module for a wearable device.

FIGS. 5A-5B show environmental views of an embodiment of a sensor for a wearable device.

FIGS. 6A-6B show an exploded view of an embodiment of a wearable device having multiple sensors.

FIG. 7 is an environmental view of an embodiment of a wearable device having multiple sensors.

FIG. 8 is an environmental view of an embodiment of a wearable device having multiple sensors.

FIG. 9 is an environmental top view of an embodiment of a wearable device having multiple sensors.

FIGS. 10A-10D show an embodiment of a wearable device having multiple sensors.

FIG. 11 is an exploded view of an embodiment of a wearable device having multiple sensors.

FIG. 12 is an environmental view in part of an embodiment of a wearable device having multiple sensors.

FIG. 13 is an environmental view in part of an embodiment of a wearable device.

FIG. 14 is an environmental view of in part an embodiment of a wearable.

FIG. 15 is an environmental view of an embodiment of a wearable device having multiple sensors.

FIG. 16 is an exploded view of an embodiment of a wearable device having multiple sensors.

FIG. 17 is a system view of an embodiment of a wearable device having multiple sensors.

FIG. 18A is a system view of an embodiment of a wearable device having data acquisition.

FIG. 18B is a schematic diagram of a monitoring and information system in accordance with the present invention.

FIG. 19 is a system view of an embodiment of a wearable device having data acquisition and/or data analytics.

FIGS. 20A-30 are flow diagrams of methods for using a wearable device.

### DETAILED DESCRIPTION OF PREFERRED EMBODIMENTS

Disclosed herein is a wearable device with one or more sensors. The device may be a wrist wearable. Sensors may be positioned about the device to measure one or more characteristics about an individual including an individual's substance use, predicted use, physiology, pathology, physical condition, mental condition, environmental surroundings, jitter, fine motor movements, and gross motor movements. The benefits and solutions can be varied and numerous. One such solution can prioritize prevention over reaction.

In one set of embodiments there is provided a device that can assist in managed care, telehealth, and/or treatment of substance abuse or addiction for individuals. Data collected from the device may be used to assist in the treatment of substance addiction, reduce the length of time for recovery, and/or reduce the time to intervention or receiving treatment. The wearable may be used to predict a person's potential for impending relapse.

In another set of embodiments there is provided a device that can be used as a deterrent to working impaired. These embodiments may help to reduce workplace accidents related to substance use, and fatigue. Certain embodiments may help change workplace behaviors and/or societal mind sets. The wearable may be worn by drivers, machine operators, and those with positions where a clear mind is needed for the safety of persons, property and the environment.

In another set of embodiments there is provided a device tied to a data collection system. Data from one or more sensors supported by the device may be used for predictive analytics. Data may be live streaming data or data at rest. The device may communicate data to a remote reporting system. The remote reporting system can empower the use of data to provide decision makers with increased transparency into their organizations, customers, and clients. A remote reporting system may assist health care providers in helping individuals through managed care, telehealth and substance abuse intervention.

The device may include a case and one or more sensors. A case is any device suitable for supporting the one or more sensors and associated electronic or electrical components (electrical). The case may include one or more seals to aid in water resistance.

The device may have electronics or electrical components connected to the one or more sensors. The electronics or electrical components may include a power source and/or conductors connected to the one or more sensors, various integrated circuits, memory, PCB, processor(s), modules, busses, connectors, boards and electrodes. The device may include one of more communication modules or interfaces. The device may have a physical, hard-wired data interface. The hard-wired data interface may be a serial port, USB port, or any other suitable communication port. The device may have a wireless input/output data interface. The wireless interface may be a radio communications module. Suitable radio communication modules include WiFi and Bluetooth modules. The data interfaces may be used for one or more of transmitting data into and/or out of the device, installing or updating firmware, installing or updating the operating system, installing or updating software or applications, transmitting data remotely, recharging a battery, or any other suitable use.

The device may include an attachment for securing the case to the user. In a wrist wearable device, the attachment may be a strap, band, bracelet, chain or other device suitable for securing the device to a user's wrist or other body part. The attachment may be associated with a case in any suitable manner. The attachment may be connected to the case or formed integral with it. The attachment may include multiple parts including one or more of: straps, closure mechanism(s), or adjuster(s). The attachment may be elastic. Elasticity may be provided in an amount sufficient to: place sensors snugly onto the user's wrists without gaps for such sensing needs; create electrical conduction with electrodes and a wear's skin; reduce ambient light exposure to the one or more light based sensors. Elasticity may be provided in an amount less than that giving discomfort to the wearer. Elasticity may be provided in an amount sufficient to allow the attachment to be fitted over a user's hand. The attachment may include multiple band parts connected to the case. The attachment may be fitted with one or more closure mechanisms. The closure mechanism(s) may be any suitable closure device including magnetic, hook and loops, buckle, tie, or any other device suitable for use as a closure mechanism.

The device may have a magnetic coupler. The magnetic coupler may be an attachment with one or more parts which can connect to the case with a coupling force sufficient to secure the device to a user during normal wear. The coupler may be configured to release upon a predetermined threshold force. The predetermined threshold may be set at a force and direction sufficient to decouple from a user below that which would injure the user should the wearable get unintendedly caught on an object. Suitable release forces may be ≥3 lbs. and ≥10 lbs. Suitable release forces may be ≤30 lbs. and ≤25 lbs.

The attachment may include electrical, such as an electrical connection, partial circuit, one or more conductors, associated electronics, or a circuit. The conductors may be provided in a wire harness. The conductor(s) may be used for connecting a power source, signal or data transmission, anti-tamper measure, powering remote sensors, indicating the device is no longer being worn, or for any other suitable means. If one or more sensors are positioned remote from the main body, remote from the case, remote from the battery or along the band, the conductors may bring power to the one or more sensors and/or provide a path for signals and data.

The attachment may include a make or break circuit. A magnetic coupler may be part of the make or break circuit. The make or break circuit may include one or more switches or connectors. In practice the circuit may indicate whether the device is removed or if there is a tamper condition. The indication may result when the circuit condition changes. A suitable circuit change may be indicated when the circuit goes from open to closed or close to open. The change condition may be activated upon the decoupling of the attachment. The change condition may be monitored by a processor. When the predetermined circuit status is detected, the processor can register the condition and/or send a signal to a remote system.

The device may include one or more sensors. The sensors may be configured on one or more electronic sensor modules. The sensor modules may include one or more sensors, front ends, amplifiers, filters, or ADC(s). The sensor module may include one or more of: an ambient light compensator, temperature compensator, humidity compensator and/or barometric pressure compensator. The sensors may be placed at any suitable location on the device, which may depend on the application, and the type of sensor. In practice and when worn by a user on the wrist, some sensors may be positioned on the upper wrist or forearm and others may be place under or the lower position of the wrist. Sensors may be placed in a case, along the attachment, or in the coupler. PPG sensors and electrochemical sensors may be placed at a location for optimized performance. Optimized performance may be realized with a tradeoff of signal strength such that each sensor will function for its intended purpose even if not maximized. A suitable place may be near or along the middle or middle ⅓ of the upper and/or lower wrist. The electrochemical and PPG sensors may be placed adjacent to each other, co-linear along the longitudinal center line of the top or lower wrist, and/or may be placed along the middle or middle ⅓ of the wrist. The electrochemical and PPG sensors may be placed at a position in the device such that when it is worn, the sensors are positioned above a higher concentration of blood vessels that that which may be found towards the outer portions of the wrist.

The device may include one or more non-invasive, alcohol sensors configured to produce an alcohol response upon activation or use. The use may be performed in any suitable manner including manual, automatic, continuous, discrete, timed, or random. Activation may be controlled by a microprocessor, analogue circuit, and/or software. A single alcohol sensor, or any number of like kind, or different kind sensors may be used as part of an alcohol detection module. The alcohol detection module may include suitable electronics, including: one or more of a front-end, amplification, filtering, feedback, potentiostat, ADC, microprocessor, power, biasing current, memory or any other suitable electronic component. The one or more alcohol sensors may be transdermal alcohol sensors. The one or more alcohol sensors may be a subdermal alcohol sensor(s). The alcohol sensor may be one or more of an electrochemical sensor, fuel cell sensor, electromagnetic sensor, optical sensor, electrochemical graphene sensor or semiconductor sensor. A suitable semiconductor sensor is a metal oxide, semiconductor sensor. A suitable electromagnetic sensor may be a light-based or optical sensor using UV, visible, infrared, near-infrared radiation, and/or Raman spectroscopy. For example, the sensor may be a photonics sensor for identifying an ethanol signal from transmitted or reflected/refracted infrared or near infrared radiation. The sensor may sense transdermal alcohol, subdermal alcohol, or both. A particularly suitable sensor is an amperometric, electrochemical gas sensor including an electrolyte, 3 electrodes in contact with the electrolyte, and one or more filters. The sensor may be configured on a module including a potentiostat.

The alcohol detection system may have a sensor with one or more of: a response time ≤15 seconds, a lower limit of detection of 0.2 ppm to 2 ppm at standard temperature and pressure; an alarm set to alert at 5 ppm to 40 ppm of alcohol vapor sensed above the skin of a human subject; a fault alarm set to alert at more than 45 ppm, more than 50 ppm or more of alcohol vapor sensed above the skin of a human subject. The fault alarm may be audible, visual and/or haptic. The alcohol detection system for monitoring alcohol may include a gas headspace at the sensor inlet. In operation the head space may be closed off by the subject's body contact during activation of the sensor.

The device may include one or more electrochemical sensors. An electrochemical sensor is a device that measures the concentration of a target analyte by oxidizing or reducing a target analyte at an electrode and measuring the resulting current. The target analyte may be a gas or liquid. The electrochemical sensor may be made up of any suitable components including: a filter stack, an electrode assembly, and an electrolyte. The electrode assembly may include at least one sensing electrode and at least one common electrode. The electrode assembly may also include a reference electrode. The electrodes may be porous and made from platinum, binder and other suitable materials. The sensor may have an electrolyte. The electrolyte may be aqueous. The electrodes may contact the electrolyte. In practice gas may diffuse to the sensing electrode at the electrolyte boundary and undergo oxidation/reduction generating current. The current may be converted to a voltage. The resulting voltage or current may be measured directly or converted to a digital form. The resulting readings may then be correlated to a predicted analyte detection or concentration.

The electrochemical sensor may have a T90 reaction time <15 seconds and more preferably a T90 time <10 seconds. The sensor may also have a recovery time <15 seconds and more preferably a recovery time <10 seconds. The reaction time and/or recovery time may be obtained at static or passive conditions such as attained without a fan or active ventilation. The times may be measured at an environmental temperature of 23 C, 1 atm, 50% RH. The sensor may be operable at a relative humidity range of 10 to 95%, a pressure range of 0.8 to 1.2 atm and/or a temperature range of −30 to 50 degrees C. The sensor may include a filter to reduce the effect of potentially interfering gases, water intrusion, or particulate matter. The filter may be a prefilter or post filter depending on the gas or analyte being detected and the type of sensor. Suitable filters may be chosen based on porosity, material reactivity, and material selectivity. The prefilter may be specific to one or may potentially interfering gases. Suitable prefilters may be selective for Carbon Monoxide, Hydrogen Sulfide, Nitric Oxide, Sulfur Dioxide, Chlorine, n-Heptane, and other Organics.

The electrochemical sensor may be placed in a sensor module. The sensor module may include a potentiostat and/or ADC. A potentiostat is electronic hardware used to control an electrochemical cell, such as a three-electrode electrochemical cell. During operation of the sensor the potentiostat may control the voltage potential between the sensing electrode and a reference electrode. This control helps to maintain a regulated system during operation. The potentiostat may then convert the resulting current to voltage. The electronics may include an ADC. The ADC may convert the voltage readings to a digital reading for the processor.

The device may include an ECG sensor. An ECG sensor or electrocardiography (ECG) sensor is a sensor system that can measure physiological parameters of an individual. The ECG sensor may be used to measure the electrical signals that control the expansion and contraction of the heart. Electrical signals may be measured by a PQRST wave form, R wave, P wave, T wave, QRS complex, PR, ST, QT, R peak, R-R peak, etc. The ECG system may be used for the detection of pace signals, lead-off detections, respiration rate, and patient impedance. The ECG measurements may be provided by a line-1 ECG electrode layout. The electrode layout may be provided by a 3-electrode system connected to module with a microprocessor. Two electrodes may be connected on one arm and one electrode connected on the other. The electrodes may be formed with any suitable conductive material. The electrodes may be made of silver, gold, conductive stainless, conductive alloys, or be plated with conductive material. The electrodes may be connected to an analogue front end or front-end amplified circuit. The analogue front end may be used to amplify and filter the signals received by the electrodes. Filtering may include one or more of a low pass filter(s), high pass filter(s), band-stop filter(s), or wavelet filter(s). The system may be configured to reject interference from strong RF sources, pace signals, lead-off signals, common-mode line frequency, signals from other muscles, and electrical noise. The filtered and amplified signals may be digitized by an analogue to digital converter (ADC). The digitized results may then be processed by a microprocessor.

The electrodes may be dry electrodes. The electrodes may be positioned on the wearable in a configuration such that the electrodes protrude sufficiently from the device to create good conductive contact with the user. One or two electrodes may be placed on top of the device and one or two electrodes may be placed below the device (between the device body and the user). The electrode contacting the skin of the wrist may be positioned in the device so when the device is worn it will generate a suitable signal to noise ratio with sufficient PQRST, QRST complex, or other wave form signal resolution. Positioning may be adjusted to optimize on a wave form particular to a specific physiological condition. Those electrodes placed between the device body and the user may be in constant contact with the user's skin. Alternatively, the electrodes facing the wrist may later meet the user's skin upon the application of pressure to the wearable.

ECG sensing may be accomplished by contacting at least one electrode with the wrist or a digit(s) of one arm and contacting at least one other electrode with a digit(s) or wrist of the other arm. An additional electrode may be positioned on either arm and contacted by one of the wrists or digit(s) of a single arm and avoiding contact with the other arms electrode (wrist or digit(s)). The ECG sensor system may be used to measure heart performance, heart metrics, heart rate (HR), heart rate variability (HRV), and other physiological parameters. HR is predominantly influenced by the coordination of the sympathetic and parasympathetic branches of the autonomic nervous system. HR may be used as an indicator of overall cardiac health. The ECG system may aid in remote health monitoring, telehealth, telemetry, or managed care applications by being part of a remote monitoring system. The ECG sensor may aid in arrhythmia detection, stress test applications, and respiration monitoring. The ECG sensor system may be used for biometric scanning for identification prediction and/or liveness testing. Biometric scanning may help to confirm the identify of a user, determine if the wearable is attached, and may be used as an anti-tamper device. The ECG sensor system may provide temporal resolution within and across days, and may be integrated into sensor fusion for improved prediction.

The device may include one or more photoplethysmography sensors (PPG). A PPG sensor is a light-based sensor system that can measure physiological parameters of an individual and certain environmental conditions. The PPG sensor may be used to measure pulse rate, heart rate variability, heart rate dynamics and recovery, blood pressure, oxygen saturation, and cardiac output. Circulatory measurements may be performed at any useful time. The PPG sensor may include one or more light emitting diodes (LEDs), one or more light sensors and a microprocessor. The LED's may be one or more of green, red or infrared light source(s). Green light may be used as providing sufficient penetration with reduced signal noise and resistance to motion artifacts even though limited by skin tone and penetration depth. Red and infrared light may have an advantage in penetrating deeper than green light into the body as it may not be absorbed as much by the skin. Light produced by the LEDs is directed at the skin, penetrates to a depth, and is directed back to a detector. The PPG sensor signal may be compensated for movement of the subject which can induce motion artifacts. The PPG sensor may also be configured for the detection or correction for ambient light condition, skin tone, tattoos, hair follicle density, and skin conditions. For blood flow monitoring and other physiological parameter monitoring the PPG sensor may be positioned on the wearable where there is a higher concentration of blood vessels over other locations in the wrist. For a wrist wearable, a suitable position may be towards the center of the wrist or over a region of high concentration of blood vessels. When the PPG sensor is used with other sensors sensor location may be optimized with tradeoffs. Circulatory measurements may be particularly beneficial when the user is at rest with the device operated at a position at approximately heart level, or during prolonged activity where heart rate is elevated. The PPG sensor system may be sensor fused with an ECG sensor system. The PPG system alone, with other sensors, or with sensor fusion may aid in detecting or predicting one or more of arrhythmia, stress, sleep quality, fatigue, respiration, substance use, substance abuse, risk of the onset or relapse of psychiatric and/or physical health conditions. The ECG electrodes may be used as part of electrodermal activity or conductance sensing.

The device may include one or more non-invasive, optical analyte sensors (NOA). A NOA sensor may be used to detect one or more analytes of interest in the human body including marijuana use, THC, opioids, morphine, fentanyl, 6-monoacetylmorphine (6-MAM), cocaine, and alcohol. The NOA sensor may have a light source. The light source may be a laser. The light source may produce one or more predetermined wavelength exposures directed at the skin of a subject. Certain wavelengths may be absorbed and/or reflected to varying degrees based on the environment, the composition of the target area, and the types and quantities of analytes present. The NOA has a detector. The detector may capture light reflected from the target area. The captured light may be used to generate a signal based on the wavelengths of reflected and absorbed light from the target based on the light impinging on the detector and/or the intensity of the wavelengths of interest. The NOA may use visible and/or near infrared light. Wavelengths of interest range from 400 nm to 1800 nm. Wavelengths and intensity of the incident ray may be determined based on the target analyte composition, subject skin composition and needed penetration depth. Analyte detection may focus on an infrared fingerprint region for the actual substance or a metabolite of interest.

The NOA may have a front end that can convert the detector signals to digital data and may transmit the data to a processor. The processor may compare the data to a library of known patterns of reflected wavelengths to predict analyte presence. The light source may have one or more filters to create the desired wavelengths of lights hitting the target. The detector may be fitted with one or more filters to limit the wavelengths it is detecting. The detector may be configured to only detect predetermined wavelengths. A processor may be used to look for the patterns of one or a select few analytes. The processor may access a digital data library of know analytes, patterns of environmental conditions, patterns of the subject without analyte, or patterns of reflected wavelengths and/or intensities to predict analyte presence or concentration. Other analyte sensors may include multichromatic sensors, UV sensors, IR sensors, infrared spectroscopy, Raman spectroscopy, mid infrared spectroscopy, and near infrared spectroscopy that may be used to determine analytes. By limiting the wavelengths of interest from one to only a few profiles, wavelengths of interest may predict analyte presence while providing a small form factor with reduced power consumption.

The device may have sensor fusion. Sensor fusion is the combination of multiple measurements from discrete sensors. The measurements may be from sensors of different types. Sensor fusion may be used to increase information, reduce uncertainty, and increase accuracy over that of any one sensor measurement or sensor type. Sensor fusion may be provided by combining the readings of two or more of any of the following: electrochemical cells, PPG readings, metal oxide sensors, ECG readings, NOA sensor(s), red light sensor(s), green light sensor(s), multichromatic sensor(s), UV sensor(s), IR sensor(s), infrared spectroscopy, Raman spectroscopy, mid infrared spectroscopy, near infrared spectroscopy, skin conductance, bioimpedance, electrodermal activity, motion sensors, temperature sensors, air quality sensors, and other current, resistive, or optical sensors. Sensor fusion may be used with one or more of artificial intelligence, predictive analysis, and neural networks for the purposes of predicting a person's mental or physical health condition, substance use, substance abuse, risk of onset or a relapse of a psychiatric and/or physical health condition. For example, a machine learning module may ingest information from physiological sensors and environmental sensors, among others, to identify risk patterns related to a desire to drink alcohol or use other substances, to make other decisions, or to provide other information or alerts. Sensor fusion combined with predictive analytics may be used in a telehealth or managed care program to aid in the intervention of addiction, recovery, relapse and support. Sensor fusion combined with predictive analytics may also be used in telematics, such as monitoring drivers and operators for possible substance use, mental awareness, or fatigue.

The device may include a biometric scanner. The biometric scanner may be an identification scanner that may evaluate the internal and/or external characteristics of a person's body to aid in identifying the user, aid in predicting user identity, control sensor activation, and/or aid in liveness testing of the user. The biometric scanner maybe one or more of a radiant energy scanner, optical scanner, capacitive sensor, an ECG device, conductive electrodes, capacitive sensor, or any other suitable device. The biometric identification scanner may evaluate a heartbeat profile or pulse profile to predict or aid in the prediction of a person's identity. A suitable scanner may be a heartbeat profile scanner which may identify certain PQRST profile patterns based off the PQRST heartbeat wave form. The profile may be created from an ECG device. The biometric identification scanner maybe one or more of a fingerprint, finger pattern or finger vein pattern scanner. The scanner may evaluate the internal and/or external surface points on a person's finger to predict identification. The finger profile may be created from a scanner as described above.

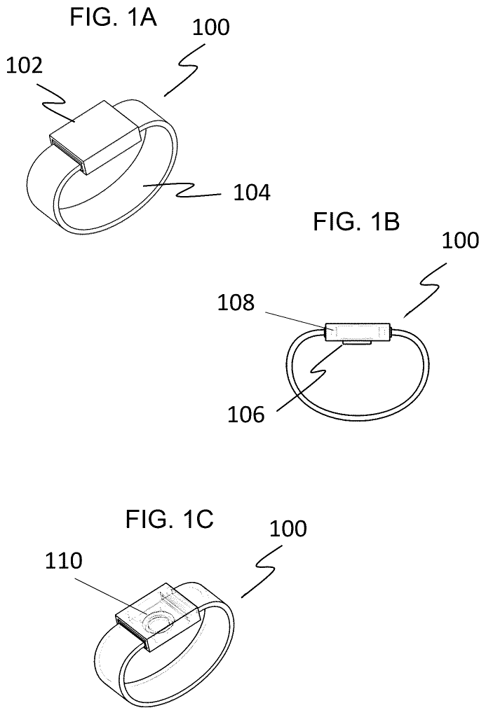

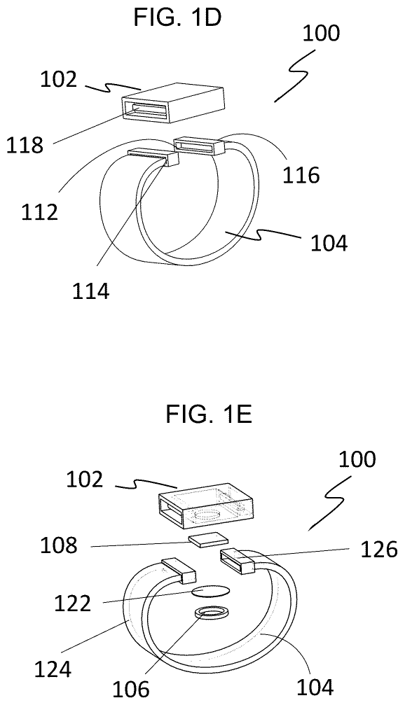

Referring now to FIGS. 1A-1E, depicted therein at 100 is a device with a substance sensor 108 supported by the device 100. The substance sensor 108 may be any suitable sensor. As show, the device 100 is a wrist wearable device with a case 102, an attachment 104, and a substance sensor 108 supported by the case 102. The attachment 104 as shown is a one-piece band with a coupler 112. The device may be worn on the wrist similar to that of a conventional watch with the case on the upper side of the wrist. Alternatively, the device may be worn with the case on the underside of the wrist. The device may include one or more additional cases and one or more additional sensors.

The sensor 108 as depicted is an alcohol sensor. The alcohol sensor is connected to electronics and a power source. The power source may be a battery. The sensor may be placed on a module and configured to produce an analyte response upon sensing the target analyte. A suitable alcohol sensor is an electrochemical, alcohol sensor. The sensor may be configured on a sensor module 110. The sensor system may also include a filter 122 and a sensor boot 106. The sensor boot may be a compressible seal between skin and wearable. The sensor boot may prevent ambient air from entering the sensing chamber or head space.

The coupler 112 may be a magnetic coupler with one or more magnets or magnetic materials 114, 116, 118. The magnetic coupler may have a coupling force sufficient to secure the wearable device to the user during normal wear but may be released upon a predetermined threshold force. The predetermined threshold may be set at a force sufficiently below that which would injure the wearer should the wearable get unintendedly caught on an object. The couple may perform more than one function, including coupling, USB connection, case attachment, conductive pathway, battery charging port, data port and others. The coupler may be sealed and/or water resistant.

The attachment 104 includes electrical 124 such as an electrical connection, partial circuit, one or more conductors or a complete circuit. The conductors may be provided in a wire harness. The conductor(s) may be used for connecting a power source, signal or data transmission, anti-tamper, powering remote sensors, indicating the device is no longer being worn, or for any other suitable means. If one or more sensors are positioned on the band or remote from the main body, the case, or the battery, the conductors may bring power to the one or more sensors and/or provide a path for signals or data.

The attachment 104 as shown includes a make or break circuit 126. The make or break circuit 126 may include one or more switches or connectors. A magnetic coupler 112 may be part of the make or break circuit. The make or break circuit 126 may have a circuit status detector to detect if the circuit has changed conditions. The circuit status detector may indicate if the device has been removed or if there is a tamper condition. The status may be determined by detecting when the circuit is has gone from high to low or low to high. The circuit may change condition upon a decoupling of the attachment 104. The changing circuit condition may be monitored by a processor, electronics, or software. When the status change is detected, the processor can register the condition and/or send a signal to a remote location.

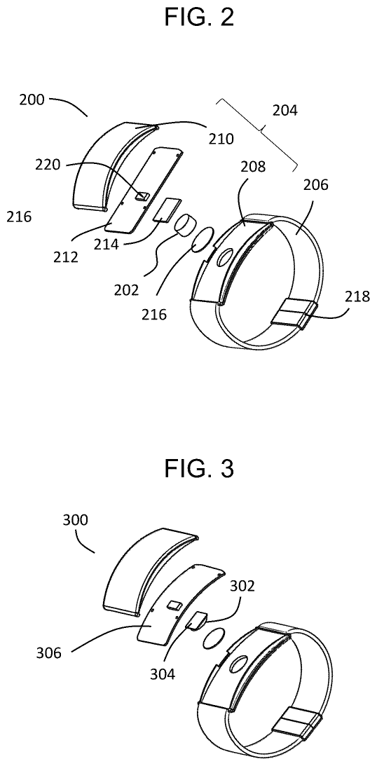

Referring now to FIG. 2, depicted therein at 200 is an embodiment of a wearable device having a substance sensor 202. As shown, the device 200 is a wrist wearable with a case 204, an attachment 206, and a substance sensor 202 supported by the case 204. The attachment is a two-piece strap or band with a coupler 218. The case 204 is multi-piece with a base 208 connected to the attachment 206 and cap 210 connected to the base. The device 200 may be worn on the wrist similar to that of a conventional watch with the case on the upper side of the wrist. Alternatively, the device may be worn with the case on the underside of the wrist.

The sensor 202 may be configured with electronics on a PCB 212, and connected to a microprocessor. The PCB may include a communications module 220. The sensor 202 may be provided on a module 214, on the PCB, or on the SBC. The sensor is powered by a power source. The power source may be a battery. The sensor module 214 may include a sensor front end and be configured to produce an analyte response upon sensing a target analyte, such as a current or voltage. The front end may include a potentiostat. The response from the sensor may be converted to digital by an ADC. The sensor 202 as depicted is an electrochemical sensor. The sensor may have a filter 216. A suitable electrochemical sensor is an electrochemical, alcohol sensor suitable for detecting transdermal alcohol.

As shown in FIG. 3, provided therein is an embodiment of a wearable device 300 having a substance sensor 302 mounted to a module 304 supported by a PCB 306. In this embodiment the PCB 306 is flexible or curved. A flexible or curved PCB may allow flexibility in case design for a better sensor fit to the user. Other features may be similar to those shown in FIG. 2.

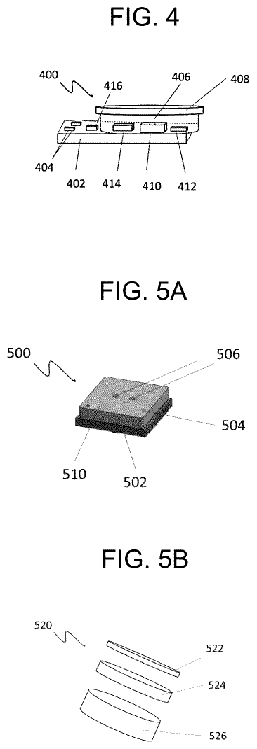

Referring now to FIG. 4, shown therein at 400 is an environmental view of an alcohol sensor module. The alcohol sensor module 400 may include an electronic circuit board 402 and associated electronics 404. The electronics may include a sensor front end, potentiostat, ADC, or other suitable electrical. The alcohol sensor module 400 may include one or more of an alcohol sensor(s) 410, a temperature compensator 412, a humidity compensator 414, and/or a barometric pressure compensator 416. The alcohol sensor 410 may be focused to a headspace 406. The headspace may include a seal 408. The seal 408 may be a flexible ring that can seal off the headspace upon contact with the user. The seal may be a flexible boot. The headspace may provide a fixed volume for transdermal perspiration or gas vapors to accumulate about the sensor 410. The humidity compensator 414 and/or the temperature compensator 412 may be located adjacent to the sensor, in the headspace, or in any other suitable location.

Referring now to FIGS. 5A-5B, depicted therein at 500 is an environmental view of an electrochemical alcohol sensor with an exploded environmental view shown in FIG. 5b showing internal components of an exemplary sensor assembly at 520. The sensor stack 510 may include one or more of: a form factor or a PCB mount 502, an electrochemical sensor 520 with electrodes, conductors connected to the electrodes of the sensor, an outer housing 504, and one or more gas inlet pores 506 through the outer housing. The electrochemical sensor 520 may include one or more components including a filter stack 522, an electrode assembly 524, and an electrolyte reservoir assembly 526. The electrode assembly 524 may include at least one sensing electrode and at least one common electrode. The sensor may include a filter to reduce the effect of contamination or interference from particulates, liquids or potentially interfering gases. The filter may be a prefilter or post filter depending on the gas and the type of sensor. The prefilter may be specific to one or more potentially interfering gases. Suitable prefilters may be selective for Carbon Monoxide, Hydrogen Sulfide, Nitric Oxide, Sulfur Dioxide, Chlorine, n-Heptane, and other Organics.

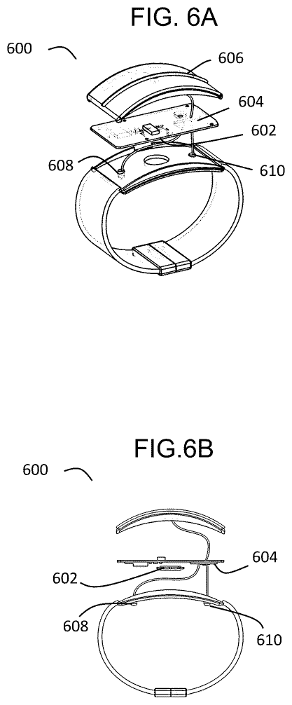

As shown in FIGS. 6A-6B, there is provided an embodiment of a wearable device 600 having a substance senor 602 and ECG module 604 with multiple electrodes 606, 608, 610. The ECG module 604 may also incorporate a PPG controller. One or more electrodes may be disposed along the top of the case. As depicted the electrode is elongated and disposed along the center region of the upper case. The elongated electrode may span substantially the entire length or width of the case from one side to the other.

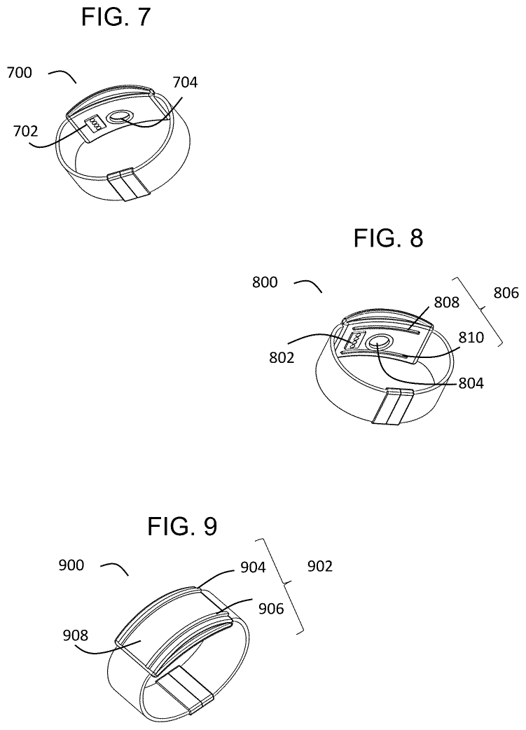

Referring now to FIG. 7 there is provided an embodiment of a wearable device 700 having multiple sensors including a PPG sensor 702, and an electrochemical sensor 704.

As shown in FIG. 8 there is provided an embodiment of a wearable device 800 having multiple sensors including a PPG sensor 802, electrochemical sensor 804 and an ECG module 806. The ECG module 806 has multiple electrodes 808, 810, one not shown.

FIG. 9 shows an embodiment of a wearable device 900 having an ECG module 902 with multiple electrodes 904, 906, one being disposed on the bottom but not shown. Electrodes 904, 906 are supported by the case 908. Two electrodes are shown extending longitudinally across the top of the case. The electrodes may extend longitudinally any length. As shown the electrodes 904, 906 extend substantially the entire length of the case parallel to each other.

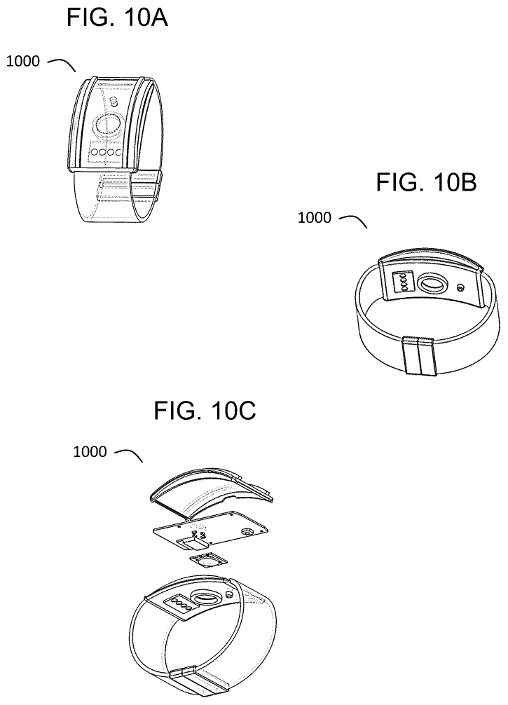

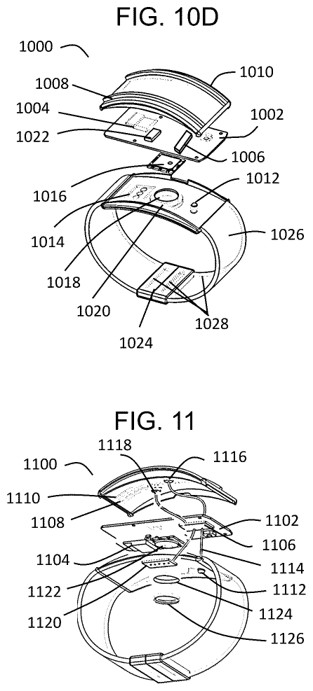

FIGS. 10A-10D, depicts an embodiment of a wearable device 1000 having a PCB 1002 with multiple sensor modules 1004, 1006. One of the senor modules 1004 may be a substance sensor module. The substance sensor module may be an electrochemical, alcohol sensor module including one or more of an alcohol sensor 1016, sensor port or head space 1018, filter, and/or a boot 1020. The device is also pictured with a PPG/ECG module 1006. The ECG has multiple electrodes 1008, 1010, 1012. Electrode wires connect the electrodes to the electronic module 1106. The device includes a PPG sensor 1014. The PPG sensor has one or more light source(s) and optical detector(s). The light source(s) and detector component(s) are connected to the ECG/PPG module 1006 with one or more conductors, such as a wire harness. The device 1000 may also include a communications module 1022. As shown the device has an attachment 1026 and a coupler 1024. The attachment includes electrical 1028 embedded within. The attachment is connected by the coupler 1024. The electrical may also be connected by the coupler 1024.

FIG. 11 depicts an embodiment of a wearable device 1100 having a PCB 1102 with multiple sensor modules 1104, 1106. The first electronic module 1104 may include any suitable substance sensor. The electronic module may be an electrochemical, alcohol sensor module including one or more of an alcohol sensor 1122, sensor port or head space 1124 and/or a boot 1126. The device 1100 is pictured with an ECG/PPG module 1106 having multiple electrodes 1108, 1110, 1112. Electrode wires 1114, 1116, 1118 connect the electrodes to the electronic module 1106. The device includes a PPG sensor 1120 with light source(s) and optical detector(s). The light sources and detector components are connected to the ECG/PPG module 1106 with one or more conductors, such as a wire harness.

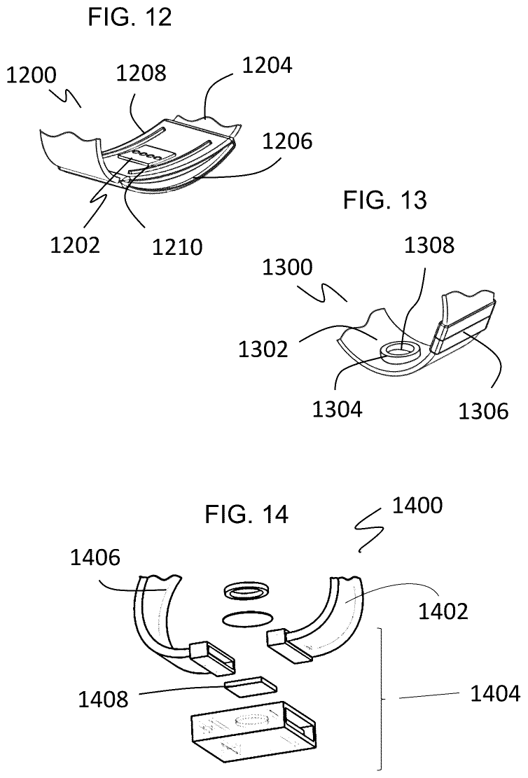

Referring now to FIGS. 12, 13 and 14, show therein at 1200, 1300, 1400 are alternative embodiments of wearables with alternative sensor positioning. The devices may be used alone or may be combined with one or more other cases to provide alternative or additional sensor coverage. Sensors 1202, 1308, 1408 may be placed or configured in any suitable position. Suitable positions include those other than above the wrist or on top of the forearm when worn by a user in a wearable application. A suitable placement may be below the wrist, outside a first case, or any other suitable position.

Pictured in FIG. 12 is an optical sensor 1202; attachment 1204 (shown in part); case 1206; first sensor electrode 1208, and second sensor electrode 1210. Electrodes 1208 and 1210 may serve a dual purpose, such as ECG, skin impedance, EDA, temperature or any other suitable dual-purpose function.

FIG. 13 shows the sensor 1308 in an attachment 1302 or band with an electrochemical sensor. The device further includes a sensor boot 1304 and a coupler 1306.

FIG. 14 shows the sensor 1408 as an electrochemical sensor in a coupler 1404. As shown the sensors are PPG 1202, ECG 1210, electrodermal activity sensor (EDA) 1208, and electrochemical 1408. The EDA sensor may measure skin impedance. The EDA sensor may share one or more electrodes with the ECG.

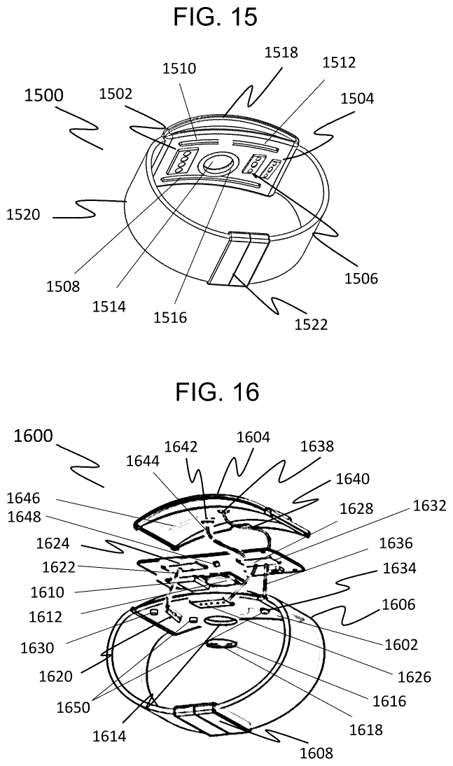

Referring now to FIG. 15, provided therein is an embodiment of a wearable device 1500 having multiple sensors 1502, 1504,1506, 1514 and an ECG module. The ECG module is shown with multiple electrodes 1508, 1510 (top electrode not shown in this view). The device includes: PPG sensor 1502, optical sensor 1504, NOA sensor 1506, electrodes 1508, 1510, skin impedance electrode (which may share electrode from ECG), temperature sensor 1512 (which may share electrode from ECG), electrochemical sensor 1514, electrochemical sensor filter and boot seal 1516, case 1518, band 1520, and a coupler 1522.

FIG. 16 shows an embodiment of a wearable device 1600 having multiple sensors 1612, 1620, 1626, 1630, 1648, 1650 and an ECG module 1632 connected to the multiple electrodes 1634, 1638,1642.

The wearable device 1600 includes: case 1602; upper case 1604; attachment 1606; coupler 1608; electrochemical sensor module 1610 with temperature, humidity, pressure sensors and/or compensation; electrochemical sensor 1612; electrochemical sensor port 1614; filter 1616; seal 1618; NOA sensor 1620; wire harness 1622; NOA sensor module 1624; PPG sensor 1626; PPG sensor module 1628; skin temperature sensor 1630; ECG sensor module 1632; lower electrode 1634; conductor 1636; upper electrode 1638; conductor 1640; upper electrode 1642; conductor 1644; extending upper electrode 1646; accelerometer & gyroscope 1648, such as a MEMS device; EDA or skin impedance sensor 1650.

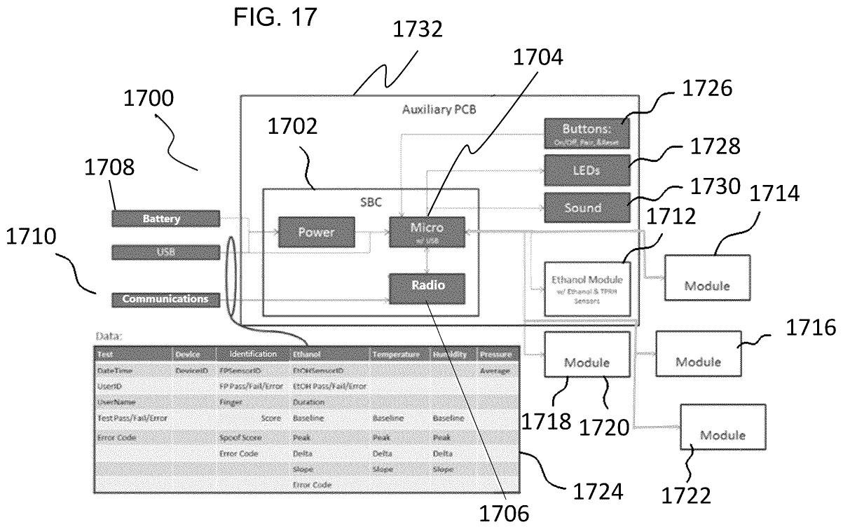

FIG. 17 depicts a system view of a device 1700 having one of more electronic and/or sensing modules. The device 1700 may include one or more of an ASIC, SBC, microprocessor or any other suitable processing device. As show the device includes a single board computer (SBC) 1702 connected to a battery 1708 and/or other suitable power source. An alternative power source may allow charging of the battery or powering the device independent of a battery and communication with the device. The single-board computer (SBC) may be a complete computing device built from a circuit board with one or more of a microprocessor(s) 1704, memory, input/output (I/O), communication, power management, and other features useful for a functional computing device. The device as shown has a communications module 1710. The communication module 1710 may provide wired and/or wireless 1706 communications. The communications module may allow the system to connect to a network or other device through USB, ethernet, radio signals, Bluetooth®, Wi-Fi or any other suitable means of connecting. The apparatus 1700 may also have data storage 1724. Data storage 1724 may include calibrations, calibration compensation, analyte libraries, applications, biometric data, user authentication, error codes, device ID, temperature, pressure, humidity, motion data, or other data. The data storage may be provided on the computing device, accessible to the processor, or accessible to an external device. Data storage 1724 may be onboard, remote or both. Data storage may be accessible using any suitable means. The apparatus may also have one or more auxiliary printed circuit boards (PCBs) 1732. An auxiliary PCB may be used for modulization or convenience in connecting peripherals, sensors or electronic modules to an SBC or microcontroller. The PCBs or modules may be connected to the SBC through one or more sockets, connectors, wire harnesses, conductors or any other suitable means. The PCB may include one or more user interfaces, including buttons 1726 and indicators 1728, 1730. Suitable input buttons 1726 include on/off, pair, and reset. The one or more PCB's 1732 may have one or more indicators. Indicators may be lights, sound 1730, or feel. Indicators may be provided with one or more LEDs 1728. Sound may be provided by speaker(s), and/or vibration devices. Feel may be provided by motion, vibration and/or haptic devices.

The device 1700 includes one or more electronic modules 1712, 1714, 1716, 1718, 1720, 1722. Suitable modules are disclosed throughout this specification. Suitable electronic modules include one or more sensing, scanning and/or substance detection modules. The modules may be selected from PPG, ECG, electrochemical, NOA sensor, biometric scanner, motion, temperature, optical, impedance, metal oxide sensor, resistive sensor, infrared sensor, and/or a biometric ID scanner. The biometric ID scanner module may be a heartbeat identification module, finger pattern detector, or any other suitable scanner. Substance sensing may be provided with a separate electronic module in electrical communication with the SBC or a microcontroller. Biometric scanning may be provided with a separate electronic module in electrical communication with a SBC or microcontroller.

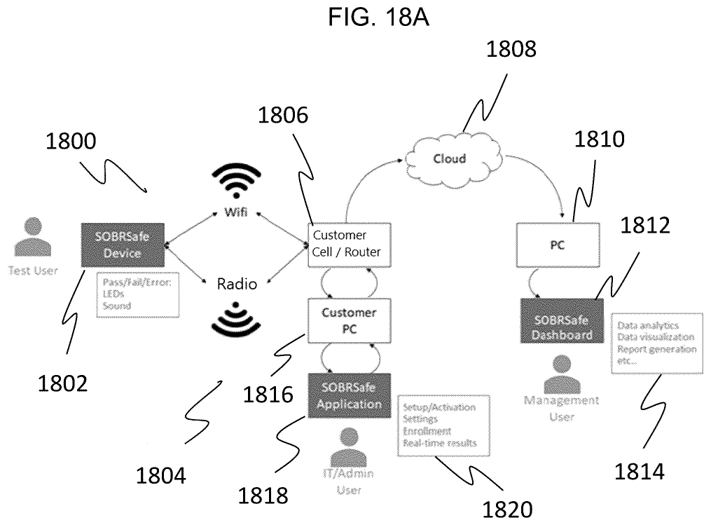

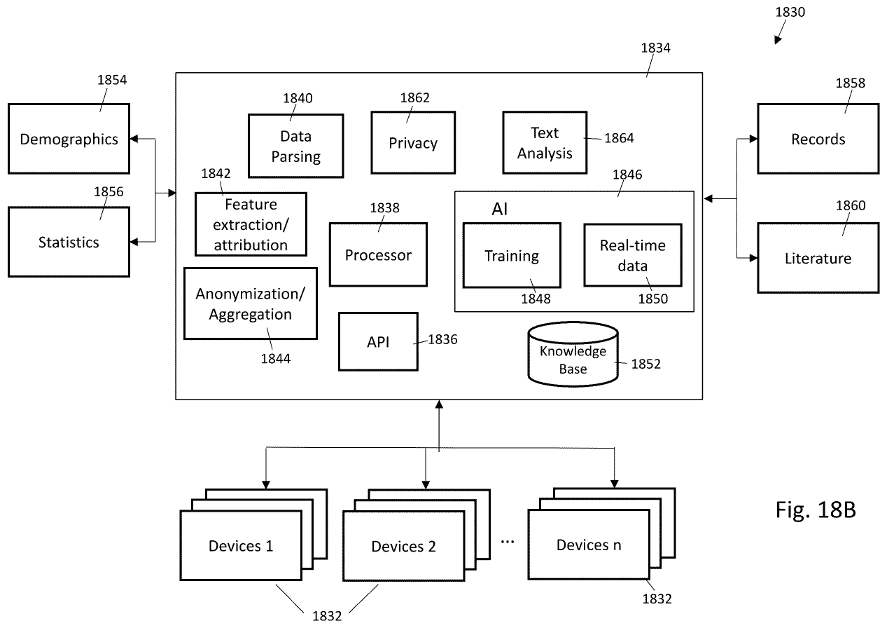

FIG. 18A shows a block diagram of a system 1800 with remote reporting. The system 1800 includes device 1802, communications link 1804, communications device 1806, cloud services 1808, user interface 1810, application 1812, database 1814, user interface 1816, application 1818, and database 1820. Each of these components is described in turn below.

The device 1802 may be any of the devices described above. In the illustrated embodiment, the device 1802 may be used, for example, by a driver of a school bus, public transportation vehicle, private ride service/taxi, or other managed fleet. The device 1802 may be used in other contexts as well such as monitoring employees in an office environment; monitoring patients or residents in an in-patient or out-patient rehabilitation facility or other support environment, monitoring driving behavior to obtain an insurance discount, or the like. As described in more detail below, the device 1802 may report to a remote platform via the application 1818 to implement a variety of functionality such as monitoring alcohol/substance use status, selectively disabling a vehicle, biometric or other identification, monitoring stress or other physiological/psychological condition, monitoring distractions, issuing alerts, and providing feedback and health/wellness information among other things. In such cases, the system users may include individual users, employers (managers, human resource professionals, administrators, etc.), fleet managers, insurance companies, healthcare professionals, sponsors and other support people, and others. Although only one device 1802 is shown for purposes of illustration, it will be appreciated that multiple devices 1802, e.g., one device per monitored user or fleet vehicle, may be employed.

There are a variety of architectures that may be employed for implementing the remote monitoring functionality. For example, an application may be hosted by an individual entity, such as a school district or ride service. Some customers may prefer this for reasons of privacy, control, and customization. Alternatively, a cloud-based platform may service multiple entities. This enables immediate access to the latest software versions and facilitates data sharing, subject to privacy controls, so as to enhance the system knowledge base and artificial intelligence/machine learning.

In the illustrated system, the device 1802 is schematically shown as communicating with processors of administrative and management users, e.g., one or more computer platforms 1810 and 1816 running the SOBRsafe application 1818 and providing dashboard 1812, via a customer cell/router 1806 and cloud-based platform 1808. As noted above, the device 1802 may include a communications module capable of wireless data network and/or Bluetooth communications. Depending on the deployment, the device 1802 may thus communicate via Wi-Fi (e.g., public Wi-Fi or a workplace network), radio (e.g., to report from the field), or other suitable network. As will be described in more detail below, the application 1818 can provide a variety of functionality relating to identification, monitoring, registration, and displaying real-time results. The dashboard 1812 may perform functions including data analytics, data visualization, and report generation among others.

FIG. 18B shows a monitoring and information system 1830 in accordance with the present invention. The illustrated system 1830 includes a number of devices 1832 that communicate with a processing platform 1834 such as a web-based platform. The devices 1832 may include devices of entities or organizations such as school districts, municipalities, employers, in-patient or out-patient rehabilitation facilities, or other entities. Moreover, the illustrated devices 1832 may include wearables or other monitoring devices as described above as well as administrative and management data terminals of the entities.

Software or other logic running on the devices 1832 may communicate (directly or indirectly) with the platform 1834 via an API 1836. For example, the API 1836 may define messaging formats, data fields, and other parameters of communications between the devices 1832 and the platform 1834. Such communications may include messages related to reports from devices 1832, queries from devices 1832, and alerts or reports to devices 1832, among other things.

The illustrated platform 1834 includes a data parsing module 1840, and a feature extraction module 1842. The data parsing module 1840 can parse information ingested by the platform 1834 from the devices 1832 and other sources, for example, to obtain certain fields of data or other sets of data. In some cases, such fields of data can be identified based on metadata associated with the data or positions of the data within headers or payloads of data streams. The feature extraction module 1842 works in conjunction with an artificial intelligence or machine learning module 1846 to extract features from the data and associate attributions with the features. For example, the module 1842 in a particular processing context, may extract information associated with analytes or combinations of analytes from one or more sensors of the devices 1832. This information may be attributed to an individual, an entity, and a location, among other things. It will be appreciated that the feature set and attributions will depend on the nature of the machine learning process and may change over time.

This information may be fed to the AI module 1846 for training and analysis. In a training process 1848, the module can process the input information to identify conditions of interest, generate alerts, and assess results as passing or failing with respect to defined criteria. In the case of supervised operation, the results generated by a data model of the may module 1846 maybe compared to results as assessed by a subject matter expert so as to provide feedback and continually train and optimize the data model. The data model may be used by a real time data module 1850 to generate results such as blood alcohol levels, substance use assessments, pass/fail assessments, generating alerts, and the like.

The anonymization module 1844 can anonymize data as desired. In this regard, it will be appreciated that information for monitoring drivers or other employees and generating alerts may need to retain an association with a specific individual for those purposes. However, the same data or other data may be anonymized, for example, to address privacy concerns. For example, information from multiple entities that is combined in a knowledge base and accessed by machine learning processes may be anonymized for such purposes. Information may be anonymized by stripping personally identifiable information or other sensitive information or aggregating information so that it loses any association with individuals.

The privacy module 1862 can store privacy preferences and settings for users and execute privacy rules for the handling of user information. In this regard, the system 1830 may allow individual users such as customers or other entities to specify what information may be used for what purposes. Thus, for example, an entity may specify that personally identifiable information may only be used for internal purposes of that entity and may allow anonymized or anonymized and aggregated information to be used for other purposes of the system 1830. In addition, privacy rules of a company may specify which users within the company can access which information and for what purposes. Thus, for example, a manager may be able to access all monitoring information, an administrator may be able to access a subset of information related to specific administrative functions, and an employee of the company may be able to access some or all of the information pertaining to that employee. All of these settings and rules can be managed by the module 1862.

As will be described below, the system 1830 may obtain some information such as records and literature in free text form, as unstructured data, or as partially structured or incomplete data. It is useful to process such information to generate, as much as possible, fully structured data including metadata for identifying data fields and attributes. The text analysis module 1864 is operative to at least partially automate this process by analyzing text in relation to content and context cues so as to extract fields of information, values, and other attributes as well as to associate metadata with the data. All of this facilitates, for example, feature extraction and attribution as well as processing by the AI module 1846.

It will be appreciated that the cloud-based platform 1834 may have access to a large volume of information concerning various monitoring environments of interest. For example, the platform 1834 may obtain information concerning various analyte measurements from various sensors, information correlating such measurements to conditions of interest, information concerning combinations of data fields such as analyte measurements and personal or demographic information of users, information correlating analyte measurements to behaviors such as driving behaviors, and many more. All of this raw and processed information may be organized and stored in a knowledge base 1852. The knowledge base 1852 can feed the AI module 1846 as well as receiving processed information from the AI module 1846 and other modules. Over time, it is expected that the knowledge base 1852 may provide unique insights into conditions of interest and concern based on the accumulated experience of the system 1830. The knowledge base 1852 may be partitioned to separate public information, private information, semi-private information, and information of particular companies or entities.

All of the modules and components of the platform 1834 may be implemented as software, firmware and/or hardware executed on the processor 1838. The processor 1838 and other modules of the platform 1834 may be embodied in a single machine or multiple machines and may be located in a single location or geographically distributed.

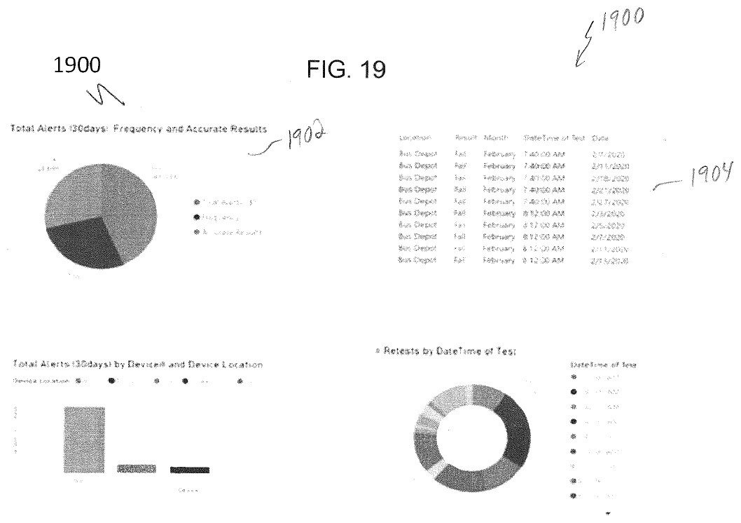

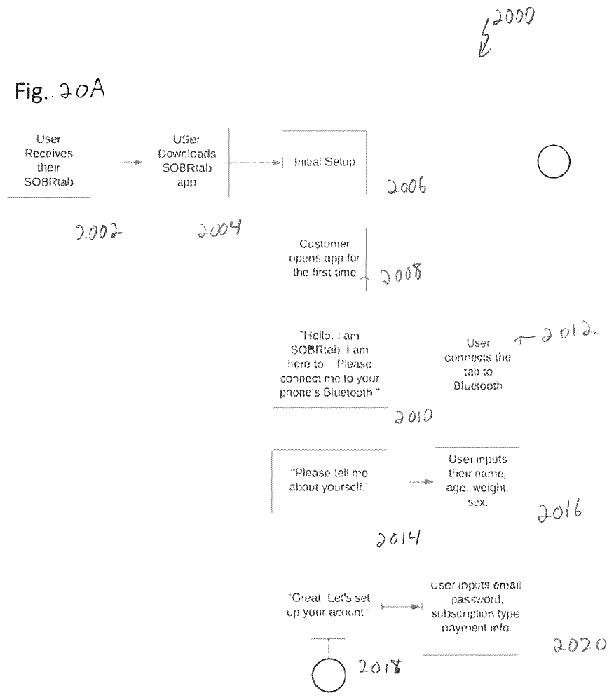

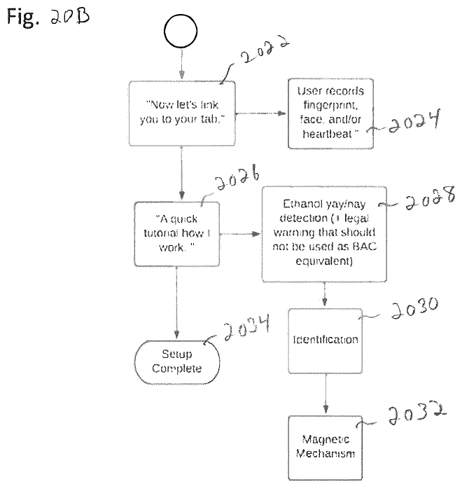

In any of these implementations, the system may provide a dashboard interface 1900 as shown in FIG. 19. The dashboard 1900 provides a quick overview or summary of information important to a company or other entity. The dashboard 1900 can be configured to provide information of interest to the entity and may include, for example, one or more fields of history, trends, analytics, or alerts for one or more of individuals, groups, or whole companies. The illustrated dashboard 1900 includes panels showing information concerning alerts 1902, scrolling information concerning recent monitoring results 1904, statistical information concerning test results by location 1906, and statistical information concerning retests by date and time 1908. The number of panels, the content of the individual panels, and the parameters used filter, sort, and display the data may all be dynamically configured and re-configured to meet the needs of each entity. The dashboard may capture data from other suitable systems, including one or more databases, applications, or cloud services.

Referring now to FIGS. 20A-30, set-up and operation routines are shown relating to a number of use cases including a general use case for individual and group users, an employment context and support contexts for individuals addressing actual or potential issues concerning alcohol consumption and other substance use. These use cases are intended to illustrate examples and are not intended to be exhaustive of the scenarios and environments where the invention may be employed. Moreover, it will be appreciated that the specific interface screens and queries set forth below represent specific implementations and the invention is not limited to those implementations. In the examples below the user device is generally referred to as the SOBRsafe tab. The SOBRsafe tab may be a wearable device as described above or other device (e.g., a desk top device or other free-standing unit) depending, for example, on the nature of the operating environment (e.g., clinical environment versus in-the-field continuous monitoring of drivers), the nature of the sensors and analytes monitored, and other factors. It will be appreciated that any of the wearables described above may include a display and speakers, as well as associated logic, for purposes of providing messages and alerts or results.

In general, the subject puts on the wearable or other user device and subscribes to an application. The identification module (e.g., biometric identification scanner) and the sensor are activated. A biometric identification scanner produces an identification response or prediction to authenticate the user. The transdermal alcohol or other substance sensor(s) produce(s) a substance prediction response proximate in time to the scanning. The device may then generate a pass-fail or risk analysis response. The response may be associated with an identification. The identification scan and the substance scan may be reported locally and/or remotely. The response may be used for any number of uses as previously described.

Referring to FIGS. 20A-20B, a set-up routine 2000 is illustrated. The routine 2000 is initiated when the users receive (2002) their SOBRsafe tabs. For example, in the case of a wearable, the user may remove the wearable from the packaging, place the wearable on their wrist or other location as appropriate, adjust the wearable for a proper fit, turn on or otherwise activate the wearable, and, in certain applications, pair the SOBRsafe tab to a mobile phone, tablet computer, or other portable data device. Once the SOBRsafe tab is activated, the user may be prompted to download (2004) a SOBRsafe app. The SOBRsafe app (or updates) may be downloaded to the SOBRsafe tab, to a mobile phone or other portable data device that is linked to the SOBRsafe tab, to a data terminal of an administrator, and/or to a data terminal of a manager, among other possibilities.

Once the SOBRsafe app is downloaded to the desired data terminals, the customer may open (2008) the app for the first time. Upon opening the SOBRsafe tab, the SOBRsafe tab or an associated data device may generate a message (2010) instructing the user to pair the SOBRsafe tab to a phone or other mobile data device via Bluetooth. For example, this message, like other messages referenced below, may be displayed on the SOBRsafe tab and/or a paired data device or other device (such as a laptop computer) used for set-up, or an audio message may be provided. The user then connects (2012) the tab to the phone or other device via Bluetooth.

The user may then receive a message (2014) prompting the user to provide personal or other user information. As noted above, the processing platform may use a variety of information for monitoring purposes including health and demographic information regarding the user. In response, the user may input (2016) a variety of information such as name, age, weight, and gender among other things. The user may then be prompted (2018) to set up an account. To set up the account, the user may input (2020) an email address, password, subscription type, and payment information among other things.

Next, the user may be prompted (2022) to link the user to the tab. It will be appreciated that establishing and verifying the identity of the user is important for security purposes as well as to ensure that accurate information is being provided. For example, it is desirable that it be difficult or substantially impossible be able to circumvent the monitoring function by allowing the tab to be used by an imposter. Accordingly, the user may record (2024) a fingerprint, facial features, heartbeat or other physiological information, or other identifying information. The user may then be prompted (2026) to view a tutorial concerning the tab and the system. The tutorial may explain (2028), for example, what alcohol or substance monitoring tests (2028) may be performed, provide any desired explanations or disclaimers (e.g., that the alcohol detection should not be used as a blood alcohol content equivalent), describe the identification process and reasons for proper identification, and explain (2032) the magnetic mechanism to connect and use the tab. Once the tutorial is completed, the set-up (2034) is complete and the tab is ready for use.

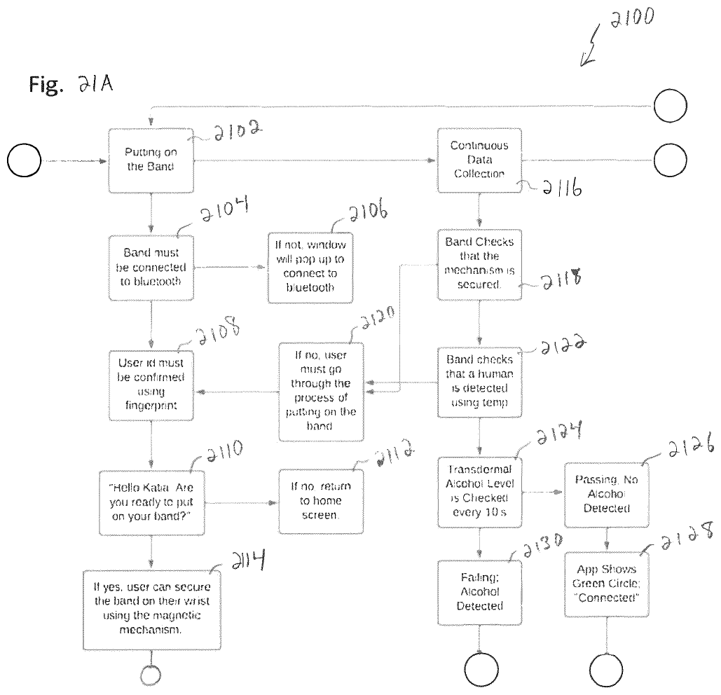

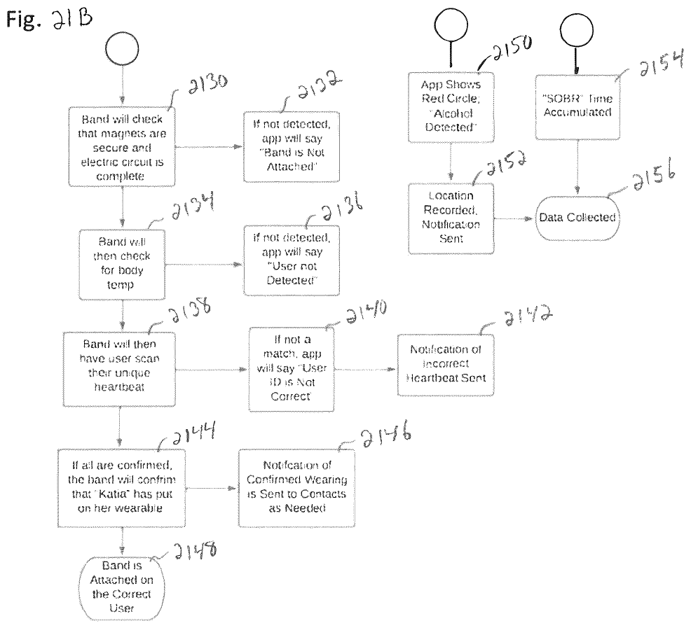

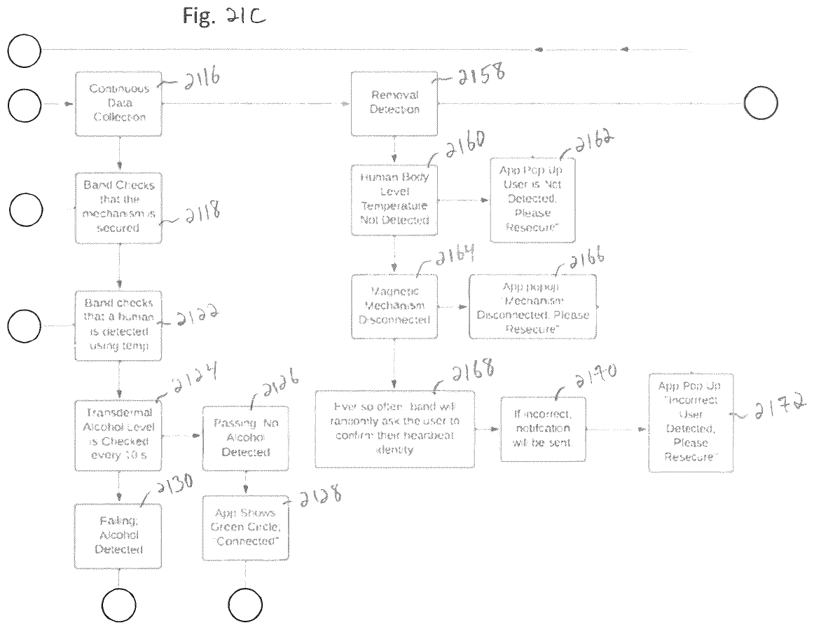

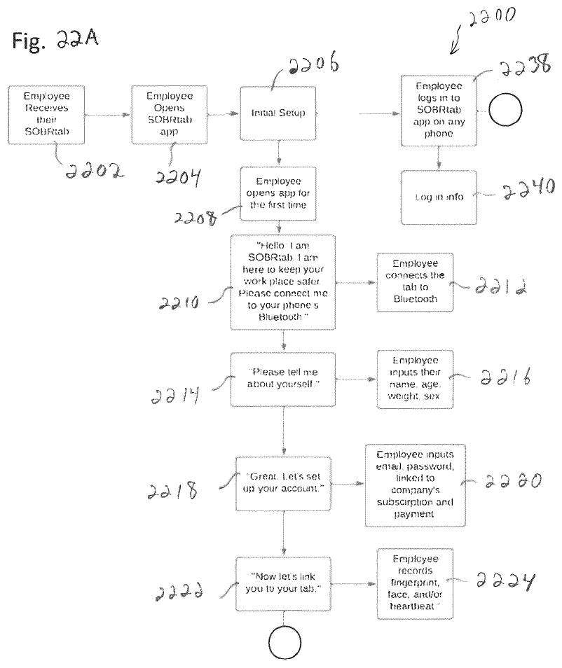

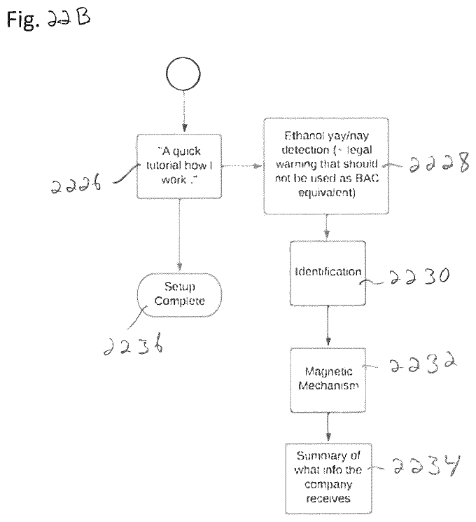

FIGS. 21A-21C show a routine 2100 for using the tab. To use the tab, the user first initiates a process (2102) for putting on the tab. In order for the tab to operate properly, the tab is connected (2104) to the user's phone or other data device via Bluetooth. If the tab is not connected, a window will pop-up (2106) to prompt the user to connect to Bluetooth. The routine when the 100 then proceeds to confirm (2108) the identification of the user, for example, by prompting the user to provide a fingerprint or other identifying information. If the identity of the user cannot be confirmed (2120), the user will be prompted to repeat the process of putting on the tab. Importantly, use of the tab cannot proceed until the identity of the user has been verified.

Once the user's identity is preliminarily verified, the user will receive a welcome message (2110), for example, asking whether the user is ready to put on the tab. If the user is not ready (2112), the routine 2100 may return to the home screen. However, if the user is ready, the user can secure (2114) the tab on their wrist using the magnetic mechanism. The tab will then check (2130) that the magnets are secure and the electric circuit is complete. If not, the app will provide a message (2132) indicating that the tab is not attached. Next, the tab may check (2134) other parameters to confirm that the tab has been attached to a live subject, that the sensors are functioning, and that appropriate readings are being obtained. For example, the app may check for an appropriate body temperature or a pulse signal (e.g., via pulse oximetry sensor readings). If these are not confirmed (2136), the app may provide a message indicating that a user has not been detected and may disable further use pending verification. The app may then prompt the user (2138) to enter dynamic identification information, e.g., to scan his unique heartbeat. If the heartbeat does not result in a match (2140), the app may provide a message indicating that the user identification has not been verified. However, if all identification processes are verified (2144), the user may be notified that the tab has been properly attached (2148) and is ready for use to begin collecting data.

The continuous data collection process (2116) then ensues. As part of this process, the tab will reconfirm (2118) that the tab is properly secured, e.g., that the magnets are secure and the electric circuit is complete. In addition, the tab may reaffirm (2112) that a live subject is detected, e.g., based on temperature and pulsatile waveform. If so, then analyte detection such as transdermal alcohol level may be checked (2124) periodically, for example, every 10 seconds or at another interval as specified by the system or selected by an entity. If the test results in a passing reading (2126), e.g., no alcohol detected, then the app may show (2128) a green circle to indicate that the application is connected and the status is acceptable. The app may then continue to monitor (2156) the subject, for example, by incrementing the accumulated time of acceptable status. If a failing reading is detected (2130), the app may show (2150) a red circle indicating that alcohol has been detected. The current location of the subject user may then be recorded (2152) and a notification may be sent, for example, to a system manager and the subject user. In addition, in certain implementations, a vehicle, workstation, or other equipment may be disabled upon detection of a failed test. The information regarding past and failed tests is collected (2156) and may be displayed on the dashboard or provided in other reports.

The app may also monitor the tab to detect (2158) removal of the tab from the user or other disabling of the tab. For example, the app may monitor human body level temperature (2160) on a continuous or periodic basis. If an appropriate temperature level is not detected (2162), the user may be prompted to resecure the tab. In addition, the app may detect (2164) that the magnetic mechanism has been disconnected or the electric circuit has been broken. In such cases, the application may provide a pop-up message (2166) indicating that the mechanism is disconnected and needs to be resecured. Moreover, from time-to-time, e.g., on a random or periodic basis, the app may prompt (2168) the user to confirm his heartbeat identity. If the identity cannot be confirmed (2170), a notification may be sent and the user may be prompted (2172) to resecure the tab.

FIGS. 22A-22B show a set-up routine 2200 for use in the context of an employee user. For example, the user may be a driver of a fleet vehicle such as a school bus driver, public bus driver, or private ride/taxi service driver. Much of the routine 2200 is similar to the routine described above in connection with FIGS. 20A-20B and those portions of the routine 2200 will only be described briefly.

The illustrated routine 2200 is initiated when the employee receives (2202) his SOBRsafe tab and opens (2204) the SOBRsafe app. The app will then proceed through an initial set-up process (2206). Set-up is initiated when the employee opens (2208) the app for the first time. The employee will then receive a startup message (2210) that may explain what the app is monitoring on behalf of the employer and why. The employee can then connect (2212) the tab to the user's phone or other mobile data device via Bluetooth. The employee is then prompted (2214) to provide personal or other user information for use by the system such as the employee's name, age, weight, and gender (2216). The employee is then prompted (2218) to set up an account and may provide inputs (2220) concerning an email address, password, and links to the employer's subscription and payment information. The employee is then prompted (2222) to link the employee to the tab. In response, the employee may record (2224) a fingerprint, facial image, heartbeat scan, or other biometric information or identification information. Users may then be prompted (2226) to view a tutorial. The tutorial may explain the tests being performed together with any desired disclaimer information (2228), the identification process (2230), the operation of the magnetic mechanism (2232), and a summary (2234) of the information that the company will receive. The employee may log-in (2238) to the SOBRsafe app on any phone or other data device by entering (2240) appropriate login information.

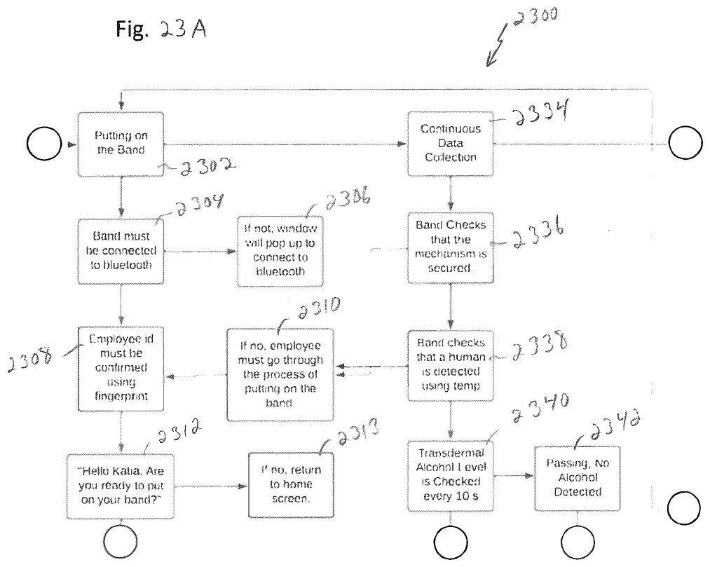

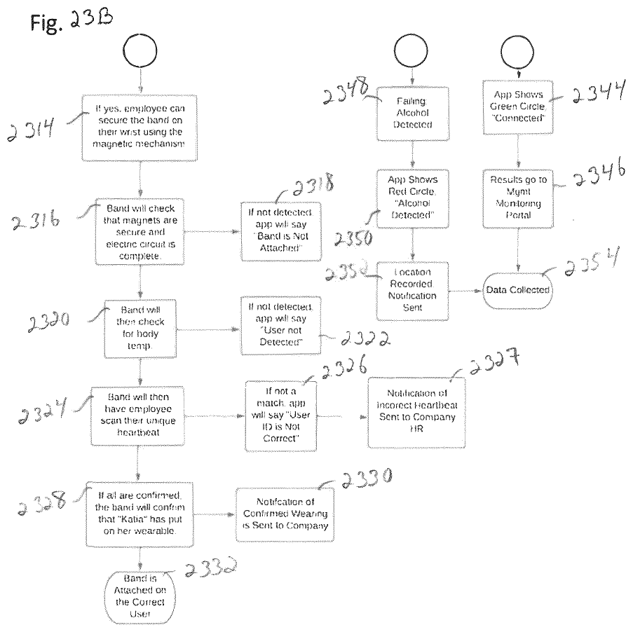

FIGS. 23A-23B illustrate a routine 2300 for using the tab in the employment context. Again, much of this routine 2300 is similar to the routine described above in connection with FIGS. 21A-21C. The routine 2300 includes a process (2302) for putting on the tab. The tab may be connected (2304) to the user's phone or another set-up device via Bluetooth. If there is no Bluetooth connection, a window will pop-up (2306) prompting the user to connect to the set-up device. The employee identification can then be preliminarily verified (2308), for example, using static identification information such as a fingerprint or other biometric information or identification information. If the identification cannot be confirmed (2310), the user is prompted to repeat the process of putting on the tab. If the employee identification is verified (2312), the app may generate a message asking whether the employee is ready to put on the tab. If not (2313), the application may return to the home screen. But if the employee is ready (2314), the employee can secure the tab on their wrist or other location using the magnetic mechanism. The tab will then check (2316) that the magnets are secure and electric circuit is complete. If not (2318), the tab may send a message to the application which will then display a message indicating that the tab is not attached. The tab can then check (2320) to confirm that a live subject is indicated, e.g., by checking for an appropriate body temperature or pulsatile waveform. If an appropriate temperature is not detected (2322), the app will display a message indicating that the user is not detected and the user will not be able to proceed with use of the system. The tab may also prompt (2324) the user to enter dynamic identification information such as initiating a heartbeat scan. If the heartbeat scan does not yield a match (2326), a message may be displayed to notify the user that the user has not been detected. In addition, a notification (2327) of the identification failure may be sent to an employer company manager, such as a huma-resources official. Depending on the system implementation, the employer may then contact the employee, disable a vehicle, workstation, or other equipment, or take other remedial action. If all identification tests are confirmed (2328), the system will confirm that the employee has put on the wearable or other user device. Such confirmation may be executed at the tab, at the application, or at a remote processing platform such as a cloud-based processing platform. Again, a notification (2330) may be sent to the employer in this regard. In certain implementations, a vehicle, workstation, or other equipment may be enabled at this point. The system then records (2332) the status of the tab as attached on the correct user.

The process (2334) of continuous data collection may then begin. In connection with this process, the system may reconfirm (2336) that the tab is properly secured to the subject employee. In addition, the system may reconfirm (2338) that a live subject is present based on, for example, temperature readings and the presence of a pulsatile waveform. If the tab is not properly secured and the presence of a live subject is not confirmed, the employee may have to repeat the process of putting on the tab. Otherwise, sensor readings (2340) such as transdermal alcohol readings may be checked on a random or periodic basis, for example, every 10 seconds. If the test indicates a passing reading (2342), the app will then show (2344) a green circle to indicate a connected status and results may be provided (2346) to management, for example, via a dashboard or other user interface screen. In addition, a vehicle, workstation, or other equipment may be enabled. In the event of a failing result (2348), the app may show (2350) a red circle indicating alcohol detected and the location of the test may be recorded (2352). In addition, a notification may be sent to management and, depending on the system implementation, a vehicle, workstation, or other equipment may be disabled. The results of all the tests may be collected (2353) together with the associated data for use in generating reports and tuning processing systems or algorithms.

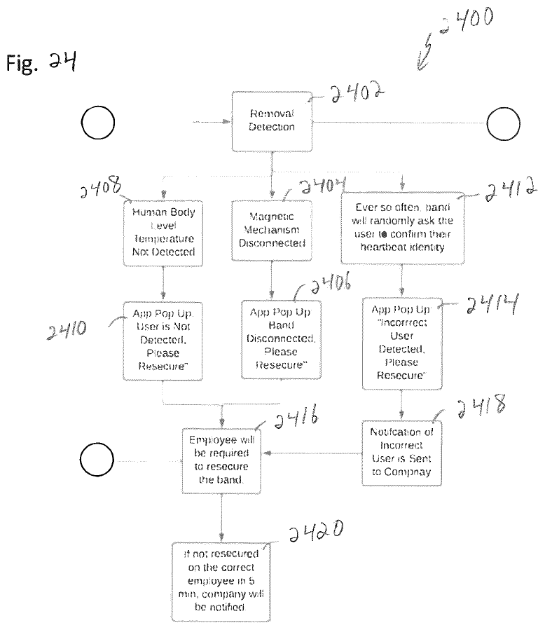

FIG. 24 shows a routine 2400 implementing a process (2402) to detect removal of the tab from the subject. As shown, removal may be detected by any of three branches. In a first branch, the system detects (2404) that the magnetic mechanism is disconnected. In response, the application may generate a pop-up message (2406) prompting the user to resecure the tab. In a second branch, the system may detect (2408) that the tab is disconnected based on a body temperature measurement or absence of a pulsatile waveform. In response, the application may generate a pop-up (2410) indicating that a user is not detected and prompting the user to resecure the tab. On a third branch, every so often, for example, on a random or periodic basis, the tab may prompt (2412) the user to confirm his identity via a dynamic identification parameter such as a heartbeat scan. If the correct user is not identified (2414), the app generates a pop-up message indicating that an incorrect user has been detected and prompting the user to resecure the tab. If the tab is not resecured within a predefined time, for example, five minutes, the company will be notified (2420). If there is a failure to confirm the body temperature or magnetic connection, the employee will be required (2416) to resecure the band. If the correct user is not verified based on the dynamic identification process, a notification of incorrect user is sent (2418) to the employer company.

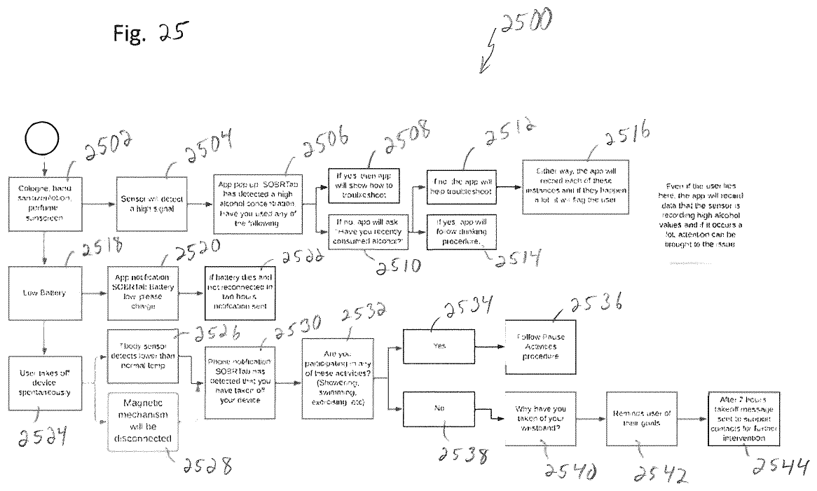

FIGS. 25-30 show routines that may be executed in connection individuals addressing actual or potential alcohol consumption or substance use issues, e.g., in an in-patient or out-patient rehabilitation process or other support settings. FIG. 25 shows a routine 2500 for addressing certain anomalies. The routine 2500 includes a process (2502) for addressing interference with readings due to cologne, hand sanitizer, perfume, sunscreens or other substances that may interfere with readings of certain analytes. In such cases, the sensor may detect (2504) a high rating due to the interfering substance. In some cases, the reading may indicate a likelihood of an interfering substance and in other cases the reading may be ambiguous. If the interfering substance causes a sensor reading indicating a failing level for alcohol or another substance (2506), the application may display a message indicating that a high reading has been obtained and may ask the user whether the user has used certain interfering substances. If the user answers yes (2508), the application can lead the user through a process for troubleshooting the erroneous measurement. If the user answers no (2510), the application may ask whether the user has consumed alcohol or used other substances. If the user then indicates that he has consumed alcohol or used other substances (2514), then the app will follow the procedure for a failed test. Otherwise (2512), the app will lead the user through a process for troubleshooting the erroneous measurement. Either way, the app will record (2516) each of these anomalies. If such anomalies are repeated or happen on a frequent basis, this may be identified so that the employee can be investigated or corrective action can be taken.

The routine 2500 also addresses low battery situations (2518). As noted above, the SOBRsafe tab may include a battery or other power source. The battery may be disposable or rechargeable. In either case, a low battery indication may be obtained from time-to-time. For example, the SOBRsafe tab may report a battery level to the paired data device and/or a remote processing platform on startup or on a continuous or regular basis. If the battery level falls below a predetermined threshold, the application may generate a notification (2520) indicating that the battery is low and prompting the user to change or charge the battery. If the battery dies (2522) and is not reconnected within a predetermined time, e.g., two hours, a notification may be sent to the employee user and/or an employer.

The routine 2500 also addresses situations where the user removes (2524) the tab spontaneously. This may be indicated by a low body temperature (2526), a disconnection of the magnetic mechanism or electric circuit (2528), or other indication of tab removal. In response, the paired device may provide a notification (2530) indicating that device removal has been detected and prompting the user to reattach the device. In addition, the app may query the user (2532) as to whether the user is participating in certain activities, such as showering, swimming, exercising, or the like, that may involve device removal or produce a device removal signal. If the user indicates that he is participating in such activities (2534), the user may be prompted to follow a procedure (2536) to pause readings. Otherwise, the user may be queried (2540) as to why he has removed the tab and may be reminded (2542) of his goals. After a predetermined time period has elapsed since the tab was taken off, a message may be sent (2544) to support contacts for further intervention.

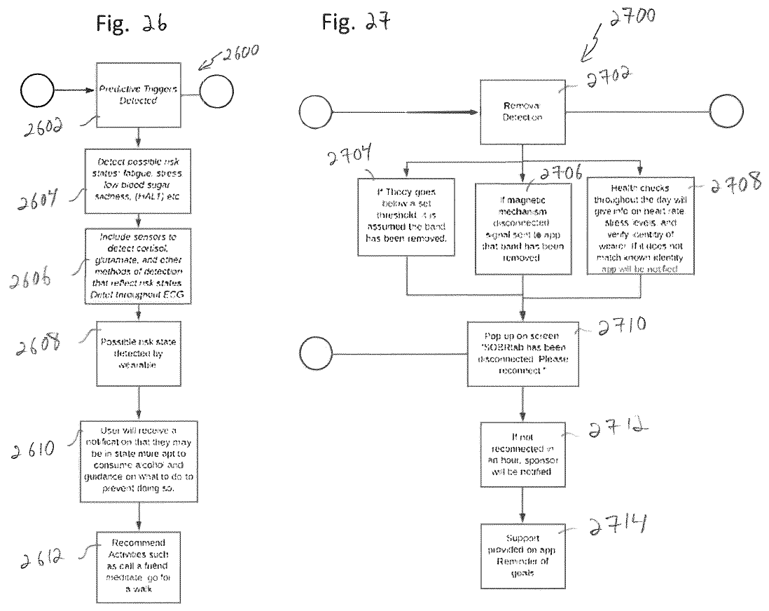

The system may also monitor and identify predictive triggers related to alcohol use or other substance use. FIG. 26 illustrates a routine 2600 that may be implemented in this regard. The routine includes a process (2602) for detecting predictive triggers. The process involves detecting (2604) certain conditions that may indicate a risk state. Such conditions may be generally predictive risk states, for example, as indicated in medical literature, relevant population risk states as indicated by literature or through mining the system knowledge base, or user specific risk states based on analysis of data for a specific user. Examples of parameters or attributes that may be analyzed in this regard include factors related to fatigue, stress, low blood sugar, mental state, and the like. These may be indicated by sensor readings, changes in sensor readings, combinations of sensor readings and external information, among other things. In this regard, the tab may include sensors (2606) to detect cortisol, glutamate, and other methods of detection relevant to risk states including ECG and brainwave analysis. Based on these readings, the system may detect (2608) potential risk states. In such cases, the user may receive (2610) a notification that they may be susceptible to consuming alcohol or using substances and may be provided guidance on what to do to prevent such use. For example, the system may recommend (2612) activities such as calling a friend, attending a meeting, meditation, or physical activity. In some implementations, a support person or group may be notified concerning the risk state.

For certain applications such as monitoring of in-patient or out-patient rehabilitation, it may be important to detect situations where the tab is removed. FIG. 27 shows a routine 2700 for monitoring removal in such contexts. The routine 2700 includes a process (2702) for detecting removal of the tab from a monitored subject. Such removal may be detected in a number of ways. If the measured body temperature goes below a set threshold (2704), the system may assume that the band has been removed. In addition, if the magnetic mechanism is disconnected or the electric circuit is broken (2706), a signal may be sent from the tab to the app indicating that the band has been removed. Moreover, the system may execute (2708) health checks that may provide information on various conditions such as heart rate and stress levels, and the system may periodically verify the identity of the wearer. If the identity does not match, or if health checks raise concerns, a notification may be generated. If tab removal is indicated by any of these processes, the app may generate (2710) a pop-up screen indicating that the tab has been disconnected and prompting the user to reconnect. If the device is not reconnected (2712) within a predetermined time, for example, within an hour, a sponsor or other party may be notified. In addition, support may be provided (2714) to the user via the app such as a reminder of goals and strategies for avoiding consumption of alcohol or use of other substances.

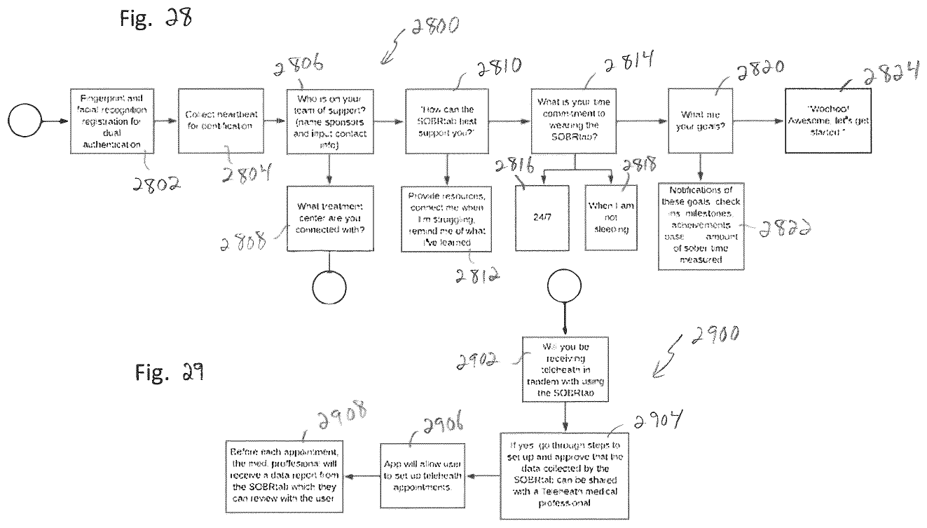

FIG. 28 shows a set-up routine 2800 that may be implemented in the context of an individual using the system for support in relation to actual or potential alcohol consumption or substance use issues. The routine is initiated by entering (2802) identification information such as a fingerprint, facial recognition information, and registration for dual authentication. It will be appreciated that accurate subject verification is critical for certain support environments. The system may then collect (2804) dynamic identification information such as a heartbeat scan, brainwave scan, or other information for active identification of the subject based on real time dynamic physiological information. It will be appreciated that such information may make it difficult or substantially impossible for a user to circumvent the system by having an imposter wear the tab. The user may then be prompted (2806) to identify who is on the user's team of support. These may be partners or sponsors in support programs or contacts at rehabilitation facilities in the case of out-patient situations. The user may be prompted to name the sponsors or other support people and provide contact information. In addition, the user may be prompted (2808) to identify a treatment center.

In addition, the system may prompt (2810) the user to provide information concerning how the system may best support the user. In many cases, users understand or have learned what situations pose risks, what strategies best support the user in achieving their goals, and what strategies are less effective. In this regard, the user may provide (2812) resources, identify which support person should be contacted in certain situations, provide reminders to the user of what the user has learned, provide reminders concerning goals, or provide other information and support. As part of the set-up process, the user may further be prompted (2814) to indicate what the user's time commitment is to wearing the tab. For example, a user may elect to be monitored (2816) 24/7, to be monitored (2818) when not sleeping, to be monitored when not at work, or to be monitored during specified intervals or to not be monitored at specified intervals. The user may further be prompted (2820) to enter the user's goals concerning the monitoring program. This may be used (2822) to provide notifications concerning the goals, to check progress towards milestones, to provide information concerning achievements, for example, based on an amount of time measured free from alcohol use or use of other substances, among other things. Once this information has been entered, the user may be notified (2824) that the system is ready to begin operation.

The system may also be used in conjunction with telehealth processes or applications. An associated routine 2900 is illustrated in FIG. 29. The illustrated routine 2900 is initiated by prompting (2902) the user to indicate whether the user will be receiving telehealth services in tandem with the system. If so, the user is prompted (2904) to go through certain steps to set-up the system and approve sharing of data from the system with a telehealth medical professional. The system may also allow (2906) the user to set-up telehealth appointments. Before each such appointment, the medical professional will receive a data report (2908) from the system for use in providing medical services to the user. In this regard, the user may determine what information may be shared with specified medical professionals and for what purposes.

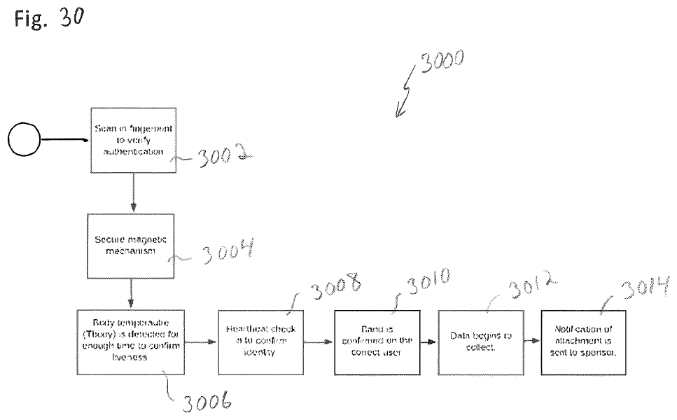

FIG. 30 shows a routine 3000 that may be implemented in connection with use of the system by an individual in a support environment. The routine 3000 is initiated by the user by entering (3002) identification information, for example, including passive and/or active identification information such as scanning in a fingerprint to verify identification. In addition, the user may secure (3004) the magnetic mechanism in connection with attaching the tab to the user's wrist or other location. The system may then be operative to confirm liveness (3006), for example, via a body temperature reading or identification of a pulsatile waveform. The system may then check (3008) the identity of the user via an active identification process such as a heartbeat scan. Through this identification process, the system may confirm (3010) that the tab is on the correct user. The system can then begin to collect (3012) data. Notifications may be sent (3014) to sponsors or other support persons concerning attachment of the tab.

As may be appreciated from the disclosure, there is provided a wrist wearable device that can better assist in the detection, prediction, screening, abstention, and/or treatment of alcohol and drug use or abuse. Also disclosed herein are embodiments of screening systems, devices, access control and methods that have one or more novel features as presented in the embodiments, claims and the figures which features may be combined in total or substituted individually. While the invention(s) has been illustrated in the foregoing description, the same is to be considered as illustrative and not restrictive in character. For example, the system of the present invention may be adapted for other uses with only slight or no modifications to the invention hereof, including as standalone device, worn on other locations, of for other living creatures. Therefore, only the preferred embodiments have been shown and described fully and that all changes and modifications that come within the spirit and scope of the claimed invention are desired to be protected.

1. A wearable device for monitoring alcohol consumption by a human subject comprising:
a band to secure the wearable device against the human subject's skin;
a gas headspace selectively closed by contact with the human subject's skin;
an alcohol sensor focused to the gas headspace to detect the presence of a target analyte within perspiration from the human subject's skin into the gas headspace, the target analyte used to detect alcohol consumption by the human subject; and
a processor to:
pair the wearable device to a remote processing platform, one or both of the wearable device and the remote processing platform storing an identity of the human subject;
detect alcohol consumption by the human subject; and
communicate the detected alcohol consumption to the remote processing platform.

2. The wearable device of claim 1, further comprising:
a biometric identification scanner to identify the human subject using one or both of internal and external surface points on the human subject's body.

3. (canceled)

4. The wearable device of claim 1, wherein the alcohol sensor includes one or more of an electrochemical sensor, a fuel cell sensor, an electromagnetic sensor, an optical sensor, an electrochemical graphene sensor, or a semiconductor sensor.

5. (canceled)

6. (canceled)

7. (canceled)

8. (canceled)

9. The wearable device of claim 2, wherein the biometric identification scanner includes one or more of a radiant energy scanner, an optical scanner, a capacitive sensor, an electrocardiography (ECG) device, conductive electrodes, or a capacitive sensor.

10. The wearable device of claim 1, further comprising:
a case enclosing the alcohol sensor and the processor; and
a sensor boot serving as a compressible seal between the human subject's skin and the case.

11. (canceled)

12. (canceled)

13. The wearable device of claim 1, further comprising:
one or more of a temperature compensator, a humidity compensator, and a barometric pressure compensator.

14. (canceled)

15. The wearable device of claim 1, wherein the processor is further to generate a fail response responsive to detecting alcohol consumption by the human subject.

16. The wearable device of claim 2, wherein the biometric identification scanner identifies one or more of the human subject's fingerprint, facial features, heartbeat, or pulse pattern.

17. The wearable device of claim 1, further comprising:
a communication module to transmit information between the processor and the remote processing platform.

18. (canceled)

19. (canceled)

20. (canceled)

21. A method for using a wearable device to monitor alcohol consumption by a human subject, the method comprising:
securing the wearable device against the human subject's skin;
detecting the presence of a target analyte within perspiration from the human subject's skin into a gas headspace of the wearable device selectively closed by contact with the human subject's skin using an alcohol sensor focused to the gas headspace;
processing information from the alcohol sensor to make a determination of the human subject's condition related to alcohol consumption; and
reporting the human subject's condition related to alcohol consumption.

22. The method of claim 21, further comprising:
pairing the wearable device to a remote processing platform;
sending information from the alcohol sensor to the remote processing platform; and
performing one or more of the identifying, processing, and reporting operations using the remote processing platform.

23. The method of claim 22, wherein the remote processing platform includes one or both of a mobile data device and a cloud-based processing platform.

24. The method of claim 21, further comprising:
identifying the human subject using a biometric identification scanner.

25. (canceled)

26. The method of claim 24, wherein the human subject's identity is based on one of a fingerprint, facial identification, heart activity, and a pulsatile waveform of the human subject.

27. (canceled)

28. The method of claim 21, further comprising:
sealing the wearable device against the human subject's skin using a sensor boot.

29. (canceled)

30. The method of claim 21, further comprising:
making a liveness determination of the human subject.

31. The method of claim 30, wherein the liveness determination is based on one or both of a body temperature reading and a pulsatile waveform of the human subject.

32. (canceled)

33. (canceled)

34. (canceled)

35. (canceled)

36. (canceled)

37. The method of claim 21, further comprising:
disabling equipment associated with the human subject in response to the report on the human subject's condition related to alcohol consumption.

38. A system for monitoring alcohol consumption by human subjects comprising:
a remote processing platform; and
a wearable device for monitoring alcohol consumption by the human subject comprising:
a band to secure the wearable device against the human subject's skin;
a gas headspace selectively closed by contact with the human subject's skin;
an alcohol sensor focused to the gas headspace to detect the presence of a target analyte within perspiration from the human subject's skin into the gas headspace, the target analyte used to detect alcohol consumption by the human subject; and
a processor to:
pair the wearable device to the remote processing platform;
detect alcohol consumption by the human subject; and
communicate the detected alcohol consumption to the remote processing platform, wherein the remote processing platform is operative to:
process information from the alcohol sensor to make a determination of the human subject's condition related to alcohol consumption; and
report the human subject's condition related to alcohol consumption.

39. (canceled)

40. (canceled)

41. (canceled)

42. (canceled)

43. The system of claim 38, wherein the remote processing platform comprises:
a biometric identification scanner to identify a human subject using one or both of internal and external surface points on the human subject's body.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)
